(19) United States
(12) Patent Application Publication
(10) Pub. No.: US 2026/0053628 A1
(43) Pub. Date: Feb. 26, 2026

(54) BONE PRESERVING ANATOMIC HIP ARTHROPLASTY SYSTEM

(71) Applicant: ABM Medical LLC, Miami, FL (US)

(72) Inventors: Roberto Augusto Miki, Pinecrest, FL (US); Ernesto Hernandez, Weston, FL (US); Scott Milton Whitten, Sunrise, FL (US); John William Box, Coral Gables, FL (US); Javier Esteban Castaneda, Miami, FL (US); Juan Sebastian Silva, Miami, FL (US)

(73) Assignee: ABM Medical LLC, Miami, FL (US)

(21) Appl. No.: 19/233,621

(22) Filed: Jun. 10, 2025

Related U.S. Application Data
(63) Continuation-in-part of application No. 18/363,572, filed on Aug. 1, 2023.

(51) Int. Cl. A61F 2/34; A61F 2/30; A61F 2/36; A61F 2/46

(52) U.S. Cl. CPC A61F 2/34; A61F 2/3609; A61F 2/4607; A61F 2/4657; A61F 2/4684; A61F 2002/30245; A61F 2002/30367; A61F 2002/30772; A61F 2002/365; A61F 2002/4659

(57) ABSTRACT

A hip arthroplasty system, intended to improve anatomic placement and maintain bone stock, includes a nail for insertion into an intramedullary canal of a femur, screw holes generally perpendicular to the nail, and a hole having a transverse axis. The system also includes a bone anchor having an opening for a cut-off femoral neck, a neck component having a longitudinal body and a seat for the femoral head, a femoral head, and an acetabular cup assembly. A jig is provided for implanting the system and includes a guide that orients relative to the nail, attachment hardware, a rail adapted to extend parallel to the transverse axis of the nail, and a second guide slidably displaceable along the rail. The system provides significant adjustability and retains maximum bone for the possibility of revision.

### CROSS-REFERENCE TO RELATED APPLICATION

This application is a continuation-in-part of U.S. Ser. No. 18/363,572, filed Aug. 1, 2023, which is hereby incorporated by reference herein in its entirety.

### FIELD OF THE INVENTION

The present disclosure relates to orthopedic prostheses. More particularly, the present disclosure relates to total hip prostheses, instrumentation therefor, and methods for performing related surgical procedures.

### BACKGROUND OF THE INVENTION

Complete hip joint replacement, or total hip arthroplasty, is the complete replacement of a damaged hip joint with a prosthetic one. The surgery is performed to relieve pain and restore function to a hip deteriorated by osteoarthritis, rheumatoid or psoriatic arthritis, avascular necrosis, congenital abnormalities or traumatic injury. Total hip arthroplasty involves replacing the entire diseased joint, composed of the natural ball and socket and its protective cartilage. The damaged joint is replaced with a prosthetic hip, usually made of a metal femoral shaft component that inserts into the femur, a metal femoral head or ball that attaches to the femoral shaft, and a metal acetabular socket, and a plastic socket liner which seats in the socket and against which the femoral head articulates.

During implantation of the femoral shaft, conventional total hip arthroplasty system may require the removal of large amounts of natural bone, often including the entirety of the femoral neck. However, allowing the patient to preserve as much natural bone as possible is viewed as a path for superior results. Natural bone has ideal mechanical properties. Further, preserved natural bone provides a platform permitting future revision surgeries, such as when a patient exceeds the wear parameters on an implant over many years of use or if an implanted system is otherwise incompatible with the patient.

In addition, conventional total hip arthroplasty systems are difficult to implant such that the femoral head and socket are properly aligned.

Also, conventional total hip arthroplasty systems require too many different components to adjust in size for different patients and are difficult to adjust to fit the patient in situ.

### SUMMARY OF THE INVENTION

A total hip arthroplasty system includes implantable components, jig components, and tool components. The implantable components include femoral side components and acetabular side components.

In an embodiment, the femoral side components include an intramedullary nail, a bone anchor, a neck component in the form a neck screw is insertable through the bone anchor, and a femoral head mountable on the neck component. The femoral side components also include fasteners including cortical screws to fix the intramedullary nail relative to the femur, anchor screws to fix the bone anchor relative to the bone of the femoral neck, and a neck component that is retained in one end of the nail.

In an embodiment, the acetabular side components include an acetabular cup, a cup liner, and fixation screws to secure the acetabular cup relative to the acetabulum.

In an embodiment, the jig components include an alignment jig with a rail and/or guide to locate and orient a cutter to remove the femoral head bone and to drill holes through the lateral and anterior cortex for cortical screws. In addition, the jig components include a reamer guide that temporarily couples relative to the bone anchor for guiding tools.

In an embodiment, the tool components include a drill bit to drill through the reamer guide and out the side of femur in one direction, and a reamer shaft with reamer head for insertion through the drilled hole and subsequent reaming of the acetabular surface to remove subchondral bone.

In accord with a surgical total hip arthroplasty procedure, the femoral head size, femoral neck length and angle, and acetabulum size are preliminarily determined. This can be performed via pre-operative planning and/or via trials upon surgically accessing the hip joint of a patient.

Then, the intramedullary canal is prepared by first preparing a hole along the axis of the canal. The intramedullary nail is provided and inserted into the hole. The nail includes an angled threaded hole having an angle conforming to a femoral neck axis, a first set of two holes for cortical screws located adjacent a distal end of the nail, and a second set of two holes for cortical screws. The second set is oriented perpendicular to the first set and located between the angled hole and a first set. The nail also includes a proximal end with a threaded opening.

The jig is coupled to the proximal end of the nail via a connector inserted at the threaded opening. In one configuration, the jig includes a rail, an anterior guide, a lateral guide attachment, and a short drill guide. The anterior guide is used to guide a drill through the second set of holes in the nail. Cortical screws are inserted through the second set of screw holes. The lateral guide and the short drill guide are used to locate a center of the femoral head, and guide a drill through a center of the femoral head. In another configuration, the jig includes the rail, the anterior guide, and a cutting guide. The cutting guide is used to guide a cutter through an intended location of the femoral neck to remove the bone of the femoral head, leaving a flat cut surface of the femoral neck.

In an embodiment, the bone anchor is a round structure having a central opening, and front and rear flat surfaces. In an embodiment, the reamer guide includes a flat head and a cannulated shaft. The head of the reamer guide is adapted to overlie a portion of the bone anchor. The cannulated shaft is threaded to engage the central opening in the bone anchor. The reamer guide is assembled through the bone anchor and together they are inserted through the drilled drill center of the femoral neck until the lower surface of the bone anchor seats on the cut surface of the neck and the shaft of the reamer guide is inserted into the angled threaded hole of the nail.

In another configuration, the jig includes the rail, the anterior guide, and a distal screw attachment attached to the anterior drill guide, and a drill guide inserted through an opening in the distal screw attachment. The drill guide is oriented to drill holes for the first set of cortical screws, and a drill bit is used to drill such holes through the drill guide. A cortical screw is inserted into one of the first set of cortical screw holes to secure the distal portion of the nail in the femur.

Then, the acetabulum is prepared. A rail connector and rail extension are coupled to the rail of the jig and the rail extension supports a drill guide. A reamer shaft is inserted through the drill guide and the reamer guide to extend in alignment with the femoral neck. A reamer head is then coupled to the reamer shaft such that the reamer head is located between the femoral neck and the acetabulum. Then, the acetabulum is reamed with the reamer head to remove the subchondral bone at the acetabulum. Then the reamer head, reamer shaft, and reamer guide are removed. Because the acetabulum is reamed with a shaft inserted through the long axis of the femoral neck and the axes of the bone anchor and angled hole, the acetabular socket is prepared.

The acetabular cup assembly is implanted into the reamed acetabular socket. If necessary, screws can be used to secure the fixation of the component. Then, an acetabular liner is inserted and secured into the component.

The reamer guide is removed and, in its place, a femoral neck component with seat is then fit into the bone anchor. The neck component can be longitudinally adjusted relative to the bone anchor. Femoral head trials are provided to the seat to confirm the appropriate size femoral head by checking stability and neck length while the trial is in place. In addition, the anteversion of the head is tested and can be varied if necessary. Once position of the components is confirmed, the trial femoral head is removed, and the position of the neck component is locked relative to the bone anchor. Then a femoral head implant matching the confirmed size of the trial is implanted on the seat of the neck component.

In another embodiment substantially similar in aspects to those described above, the femoral side components include an intramedullary nail, a bone anchor, a neck component in the form of a neck beam, and a femoral head mountable on the neck beam. The nail has an angled neck hole for the neck beam. The neck beam and neck holes are non-circular in cross-sectional shape, and more preferably have narrow tall shapes. In a preferred embodiment, the neck beam and opening defines triple barrel shapes. These features increase the strength of both the intramedullary nail and the neck beam in bending. The neck beam does not rotate relative to the intramedullary nail but has features that permit discrete axially offset positions within the opening in the nail. The femoral components described above can be assembled to the neck beam and used with this embodiment.

The systems allow minimal bone resection and accurate restoration of the native joint anatomy, while providing excellent intraoperative alignment and adjustment. The systems allow preserving a maximum portion of the femoral neck and the proximal femur bone stock. The systems allow reconstruction of natural hip biomechanics. The system allows load sharing through the entire proximal femur. The systems provide the ability to have the acetabular cup and femoral neck in perfect alignment. The systems provide the ability to adjust the femoral neck length after reduction through the femur both laterally and medially. The systems allow final adjustment of the neck length in situ. The systems allow accurate replication of natural anteversion of the joint and the ability to adjust anteversion intraoperatively. The systems facilitate later revision without bone resection greater than presently required for current total hip arthroplasty systems.

### BRIEF DESCRIPTION OF DRAWINGS

FIG. 1 is an assembly view of components of a total hip arthroplasty system.

FIG. 2 is a view of the total hip arthroplasty system overlaid against a natural femur.

FIG. 3 is a perspective view of an intramedullary nail component of the system.

FIG. 4 is a first side elevation of the nail component of FIG. 3.

FIG. 5 is a second side elevation perpendicular to the first side elevation in FIG. 4.

FIG. 6 is an enlarged top view of the nail component of FIG. 3.

FIG. 7 is an enlarged bottom view of the nail component of FIG. 3.

FIG. 8 is a perspective first partial assembly view of a jig relative to the nail component.

FIG. 9 is a perspective second partial assembly view of a jig relative to the nail component.

FIG. 10 is a perspective third partial assembly view of a jig relative to the nail component.

FIG. 11 is a perspective view of the jig with apex ring relative to the nail component.

FIG. 12 is perspective view in the anterior to posterior direction of the jig and nail component relative to a femur.

FIG. 13 is perspective view in the medial to lateral direction of the jig and nail component relative to a femur.

FIGS. 14 through 16 illustrate steps of a method described herein.

FIG. 17 is a plan view of a bone anchor component of the system.

FIG. 18 is a perspective view of the bone anchor component of the system.

FIG. 19 is a plan view of a reamer guide component of the system.

FIG. 20 is a perspective view of the reamer guide component of the system.

FIGS. 21 through 28 illustrate steps of the method described herein.

FIGS. 29, 30, 31, and 32 are perspective, front, side and rear views, respectively, of a reamer tool described herein.

FIGS. 33, 34, 35, and 36 are perspective, front, side and rear views, respectively, of a reamer adapter described herein.

FIG. 37 illustrates a step of the method described herein.

FIGS. 38, 39, 40 and 41 are perspective, inside, side, and outside views, respectively, of an acetabular cup component of the system described herein.

FIGS. 42 and 43 illustrate steps of the method described herein.

FIGS. 44, 45, 46 and 47 are perspective, inside, side, and outside views, respectively, of an acetabular cup liner component of the system described herein.

FIGS. 48 and 49 illustrate steps of the method described herein.

FIGS. 50 and 51 are side elevation and perspective views, respectively, of a neck screw of the system described herein.

FIGS. 52 and 53 illustrate steps of the method described herein.

FIGS. 54, 55, 56 and 57 are medial, lateral, anterior and perspective views, respectively, of a trial femoral head of the system described herein.

FIGS. 58 through 61 illustrate steps of the method described herein.

FIGS. 62, 63, and 64 are medial, anterior and perspective views, respectively, of a femoral head of the system described herein.

FIGS. 65 and 66 illustrate steps of the method described herein.

FIGS. 67 and 68 illustrate a cutting gauge and steps for use in association with the system described herein.

FIG. 69 is a perspective exploded view of a reamer guide and bone anchor insert according to a first alternate embodiment of the system.

FIG. 70 is a perspective assembly view of the reamer guide and bone anchor insert of FIG. 69.

FIG. 71 through 76 illustrate steps of an alternate method using the reamer guide and bone anchor insert of FIGS. 69 and 70.

FIG. 77 is an assembly view of components of the first alternate embodiment of the system described herein.

FIG. 78 is a side elevation partial assembly of a second alternate embodiment of the system.

FIG. 79 is a view similar to FIG. 78, rotated 90° and tilted so that the neck beam is horizontal.

FIG. 80 is a perspective view of the intramedullary nail of the second alternate embodiment of the system.

FIG. 81 is a perspective view of a neck beam in the second alternate embodiment.

FIG. 82 is an end view of the neck beam of FIG. 79.

FIG. 83 is an exploded view of an assembly of the neck beam and a bone anchor washer.

FIG. 84 is an assembled view of the neck beam and a bone anchor washer.

FIG. 85 illustrates implantation of the neck beam and bone anchor washer at the cut end of a femoral neck.

FIG. 86 illustrates alternate methods of adjusting the neck beam relative to the nail when trialing a trial femoral head, using a driver coupled at either end of the neck beam.

FIG. 87 is a longitudinal section through an assembly similar to FIG. 78, without the femoral head, illustrating the neck beam locked in position in the intramedullary nail by a set screw assembly.

FIG. 88 is an enlarged side elevation view of the set screw assembly shown in FIG. 87, rotated 90°.

FIG. 89 shows an exploded view of the set screw assembly of FIG. 88, including a lower tip and an upper base which can rotate relative to the tip.

FIG. 90 is an assembly view of components of the second alternate embodiment of the system.

### DETAILED DESCRIPTION

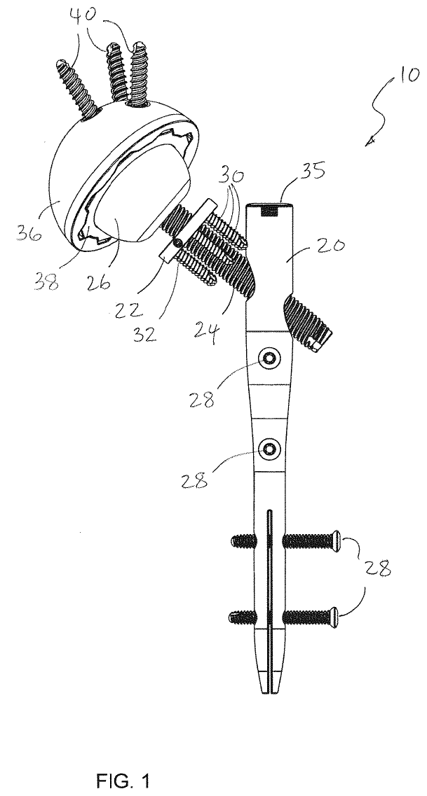

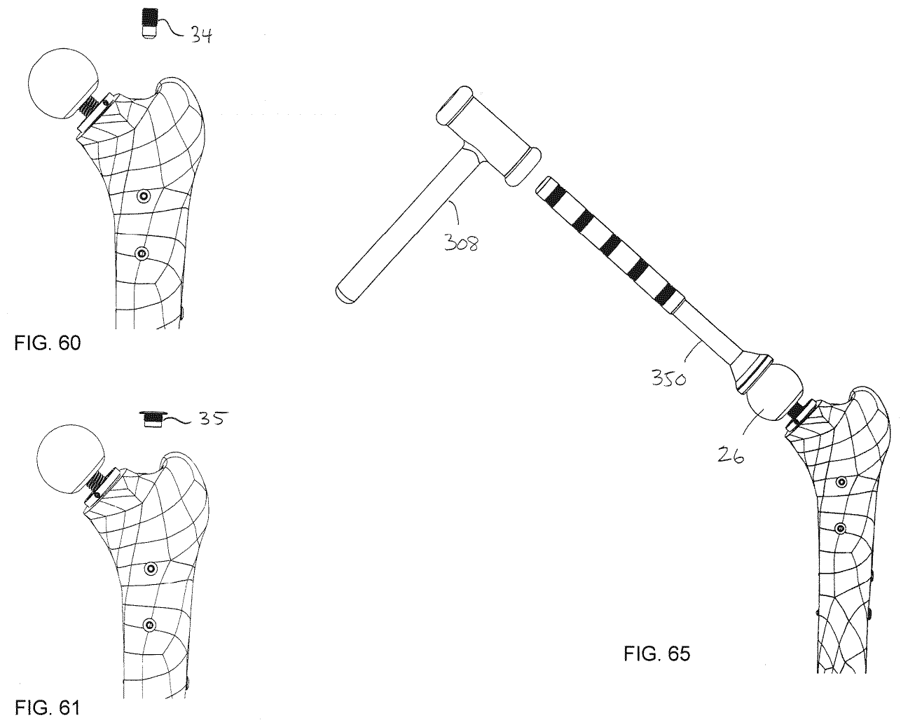

Referring to FIG. 1, a total hip arthroplasty system 10 includes femoral side implantable components and acetabular side implantable components. The femoral side components generally include an intramedullary nail 20, a bone anchor in the form of a nut 22, a neck component in the form of a neck screw 24 insertable through the bone anchor nut 22, a femoral head trial 25 temporarily mountable at one of the neck screw 24, and a femoral head 26 permanently mountable at one end of the neck screw 24. The femoral side components also include fasteners including cortical screws 28 to fix the intramedullary nail 20 relative to a femur, and a nail set screw 34 (FIG. 60) insertable in a proximal end of the nail 20, and an end cap 35. These components will all be described in more detail below in association with other components, methods and various preferred and optional techniques.

The acetabular side 14 components generally include an acetabular cup 36, fixation screws 40 adapted to secure the acetabular cup in the acetabular socket, and a polymer cup liner 38 preferably made from an ultra high molecular weight polyethylene (UHMWPe). All of the implantable components other than the cup liner 38 are preferably made from a suitable metal such as titanium, cobalt chromium, or stainless steel and optionally may be coated or treated for additional wear resistance or bone ingrowth. These components are all described in more detail below.

The system also includes reusable or disposable trial components. For example, the system may include a plurality of nails trials of different lengths, femoral head trials of different diameters, and neck screw trials of different lengths.

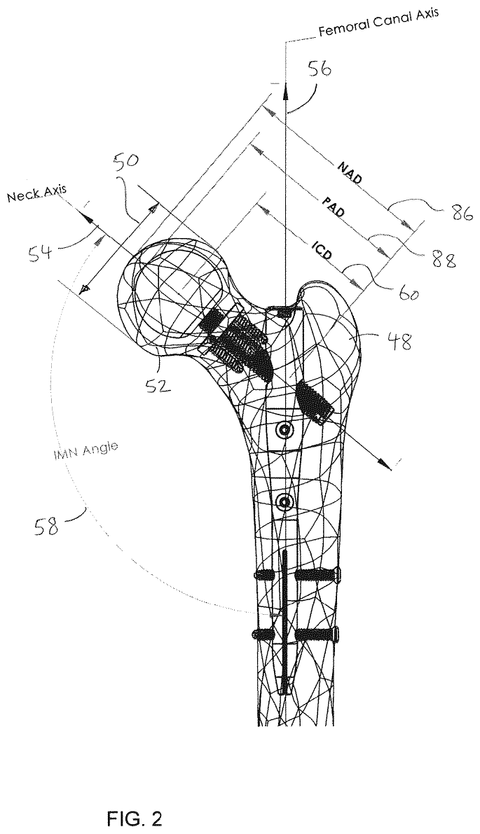

Referring to FIG. 2, the femur 48 is prepared to receive the intramedullary nail 20. An incision is made in a suitable approach, for example from a typical anterior hip Smith-Peterson approach. In such an approach, an incision is made over the anterolateral portion of the hip. The lateral femoral cutaneous nerve is protected. An interval between the sartorius and the tensor fascia lata is created. Deep dissection is continued to expose the anterior capsule of the hip. The rectus femoris muscle may be detached from its origin off the acetabulum. The lateral femoral circumflex vessels may be ligated or cauterized. The anterior capsule is incised and may be removed. The femoral head is exposed and dislocated using any suitable technique. Then, a guide pin is attached to entry portal instrumentation and a K-wire is inserted, preferably 2 to 3 cm, into the piriformis start point. A preferred entry point for the K-wire is located on the medial facet of the greater trochanter, 4° from the anatomical axis in the anterior-posterior (AP) view, and aligned with the femoral canal axis 56 in the lateral view. The K-wire is advanced under fluoroscopic guidance to below the level of the lesser trochanter. A nail reamer is advanced over the K-wire below the level of the lesser trochanter. The nail reamer and K-wire are then removed from the femur 48.

In accord with a surgical technique for total hip arthroplasty, preoperative planning initially determines several anatomical measurements relative to the operative femur 48. The measurements include determining the diameter 50 of the native (or natural) femoral head 52, identifying a central neck axis 54 of the native femoral head 52, determining the femoral canal axis 56, estimating the femoral neck angle 58 of the neck axis 54 to the femoral canal axis 56 (referred to herein as an IMN angle), and measuring a distance 60 from the center of native femoral head 52 to the femoral canal axis 56 along the femoral neck axis 54, referred to as the Intersection-Center Distance (ICD). The femoral neck angle 58 is preferably selected from a preselected group of angles between 110° and 140° and more preferably at, for example 120°, 125° and 130°.

Based on the selective anatomical measurements, implants are selected. The acetabular cup is selected from various sizes of acetabular cups based on the native femoral head diameter 54. The cup liner and femoral head are selected from various sizes of cup liners and femoral heads based on the acetabular cup selected. The intramedullary nail is selected based on the angle 58 of the femoral neck. The length of neck screw selected is based on the Intersection-Center Distance (ICD) 60.

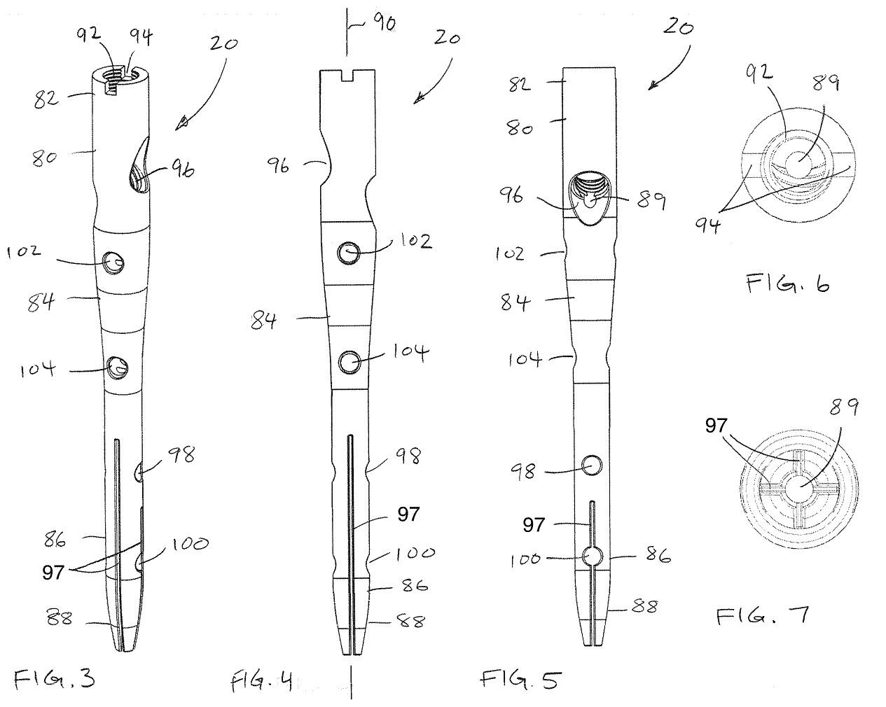

Referring to FIGS. 3 through 7, the intramedullary nail 20 is a preferably unitary rod having a proximal portion 80 having a proximal end 82, a central portion 84, and distal portion 86 having a distal end 88. The nail 20 is cannulated with a central bore 89. The nail 20 defines a straight longitudinal axis 90 that is adapted to extend parallel to the femoral canal axis 56 (FIG. 2). The proximal end 82 of the nail is flat, includes a threaded bore 92, and has a transverse registration slot 94 that extends through the threaded bore 92. An angled threaded bore 96 having an axis corresponding to the IMN angle 58 is provided in the proximal portion 80. The distal portion 86 has a crosswise split 97 along the longitudinal axis 90 and includes a first set of screw holes 98, 100 having a first pair of axes that extend across the split 97; i.e., through the longitudinal axis. The distal portion 86 is tapered at the distal end 88. The central portion 84 includes a second set of central screw holes 102, 104 having a second pair of axes that extend perpendicular to the axes of the distal screw holes 98, 100 and perpendicular to the longitudinal axis 90. The central portion 84 is also tapered in diameter between the two central screw holes 102, 104. The proximal portion 82 and a portion of the central portion 84 of the nail are preferably textured, e.g., by sintering, to enhance bone ingrowth. The nail 20 has a substantially circular cross sectional shape transverse to the longitudinal axis along its length from the proximal end 82 to the distal end 88. “Substantially circular” with respect to the shaft of the nail is defined to mean circular, excepting any inconsistencies that would be presented in the circumferential shape resulting from the provision of screw holes, bores, or splits as present in the nail 20.

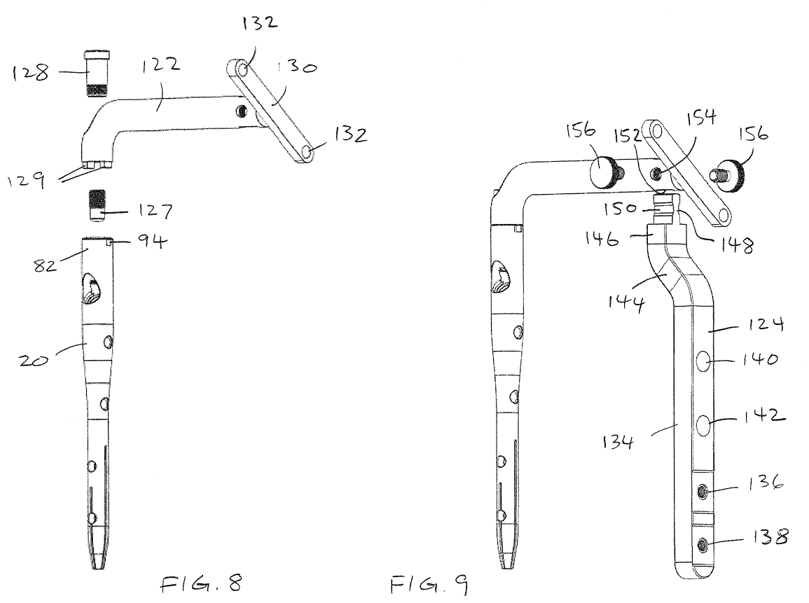

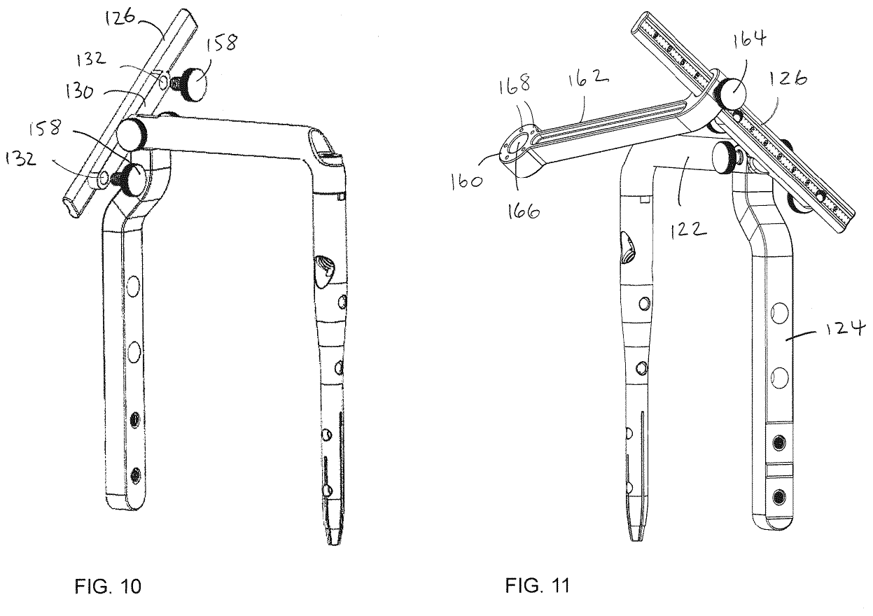

Turning now to FIGS. 8 through 11, an implantation jig 120 is provided for guiding screws into the nail 20, orienting instruments, aiding in removal of femoral head bone, guiding implantation of the acetabular cup 36 (FIG. 1), and otherwise aiding in implantation and orientation of the prosthesis system 10 (FIG. 1) described herein. The implantation jig 120, in a basic configuration, includes an elbow component 122, an anterior guide 124, and a rail 126. The elbow component 122 is attached to the proximal end 82 of the intramedullary nail 20 with a neck set screw 127 and base screw 128. One end of the elbow component 122 has two feet 129 rotationally fixed at the transverse registration slot 94. The other end of the elbow component 122 includes a mounting bracket 130 oriented at a defined angle and including mounting holes 132 adjacent its ends. Referring to FIG. 9, the anterior guide 124 has a first straight portion 134 with two lower threaded holes 136, 138, two central non-threaded holes 140, 142, a bent portion 144, and a second straight portion 146 parallel to the first straight portion. A thin arm 148 protrudes axially from the second straight portion 146 and includes grooves 150 on opposite sides thereof. The thin arm 148 fits within a slot 152 in the elbow component 122, and two threaded screw holes 154 communicate with the slot 152. Thumb screws 156 extend within the screw holes 154, seat within the grooves 150 on opposite sides of the arm 148 and secure the anterior guide 124 to the elbow component 122. When so secured, the anterior guide 124 extends parallel to the nail 20, with the non-threaded holes 140, 142 aligned with the central screw holes 102, 104, and the threaded holes 136, 138 at the same longitudinal location as the distal screw holes 98, 100, but oriented in a transverse orientation. Turning to FIG. 10, the rail 126 includes screw holes (not shown) and is coupled to the bracket 130 with two knob screws 132 inserted through the mounting holes 132 of the mounted bracket 130. The rail 126 preferably includes a measurement scale via indicia printed, etched or otherwise visible thereon.

Referring to FIG. 11, in one configuration of the jig 120, an apex ring 160 is provided. The apex ring 160 includes an arm 162 slidably coupled to the rail 126 and thumb screw 164 to fix the location of the arm 162 relative to the rail 126. The ring 160 includes a central opening 166, and preferably a plurality of holes 168 for receiving K-wires. The apex ring 160 may be referenced relative to the anatomy, temporarily fixed relative to the anatomy, and drilled through. In one procedure, the apex ring 160 is positioned a specific distance relative to the native femoral head 52. In a preferred method, the defined distance is determined as the ICD+the native femoral head diameter/2, and is referred to as Native Apex Distance (NAD) 86 (See FIG. 2). The dimensions are obtained from patient imaging. The imaging may include x-rays, magnetic resonance images (MRI), nuclear imaging, fluoroscopic imaging or other suitable imaging that provides sufficient resolution of the bones of the hip joint. The location of the apex ring 160 relative to the measurement scale of the rail 126 when registered relative to the prosthetic femoral head is determined as (ICD+the prosthetic femoral head diameter/2), and is defined as Prosthetic Apex Distance (PAD) 88.

Once the jig 120 as described is assembled to the proximal end of the nail 20, the nail 20 is ready to be advanced into a drilled hole in the proximal femur. A hole is drilled through the proximal femur and into the femoral canal along the femoral canal axis in any conventional manner. The nail 20 is inserted into the femoral canal along the femoral canal axis 56 (FIG. 2). Turning to FIG. 12, the apex ring 160 is positioned on the rail 126 at the Native Apex Distance 86 (FIG. 2), determined from the preoperative planning. The jig 120 is used to locate the apex 170 of the femoral head 52, defined as the most medial point of the longitudinal axis of the femoral neck. The apex ring 160 may be positioned on the apex 170 of the femoral head by rotating the nail 20, adjusting the depth of the nail, and/or sliding the apex ring 160 relative to the rail 126. The apex ring 160 is locked in position relative to the nail by tightening the associated set screw 164.

Then fluoroscopic images of the nail 20 are obtained to confirm the location of the nail within the proximal femur, and any necessary adjustments are made. Once the location is confirmed, smaller incisions are made in the fascia and a drill sleeve (not shown) is inserted through the incision into one of the first set of screw holes 140 in the anterior guide 124, and a hole is drilled with a drill. A cortical screw 172 is inserted through the drilled hole and the nail 20 to longitudinally and rotationally fix the nail 20 relative to the femur 48.

Referring to FIG. 14, a drill guide 174 is then inserted through the apex ring 160, and a drill 176 is used to drill through the guide 174 and apex ring 160, the natural femoral head 52, the angled hole in the nail 96 (FIGS. 3, 4, and 5), and the endosteal surface 178 of the far cortex of the femur 48 (but preferably not through the cortex). The set screw 164 for the apex ring 160 is then loosened and the apex ring 160 is removed from the jig 120.

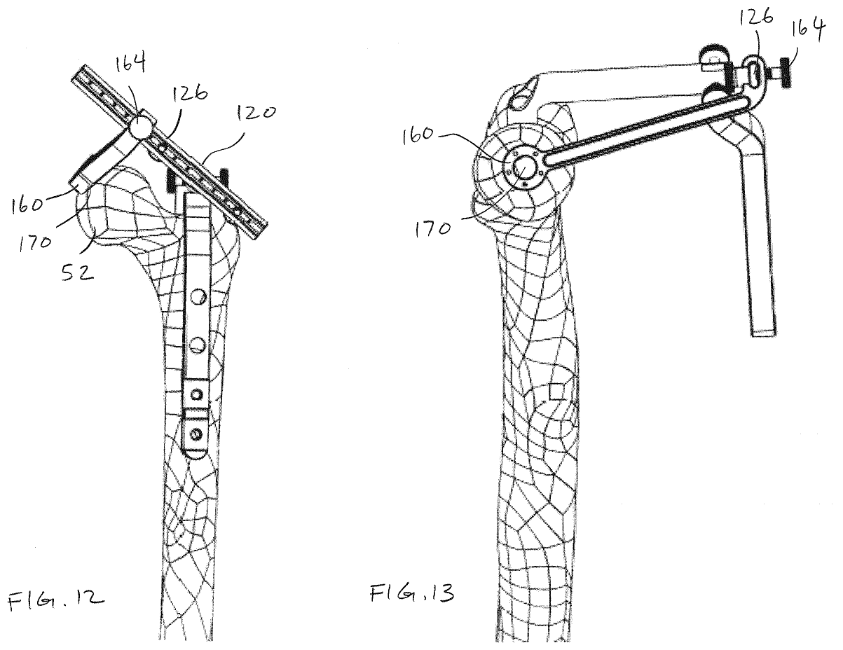

Turning to FIG. 15, a cutting guide 180 is then attached and secured to the rail 126 of the jig 120. The cutting guide 180 defines a slot 182 for an oscillating saw blade and/or cutter relative to the femoral neck 184 so that the cutter can remove the natural femoral head 52. The location of the cutting guide 180 is preferably based on a femoral neck length that maximizes bone stock without causing impingement and is set between the native femoral head base and the isthmus of the femoral neck. As shown in FIG. 16, the natural femoral head 52 (FIG. 13) is then cut off from the femur 48 using the cutting guide 180. The cutting guide 180 is then loosened and removed from the rail 126 of the jig 120.

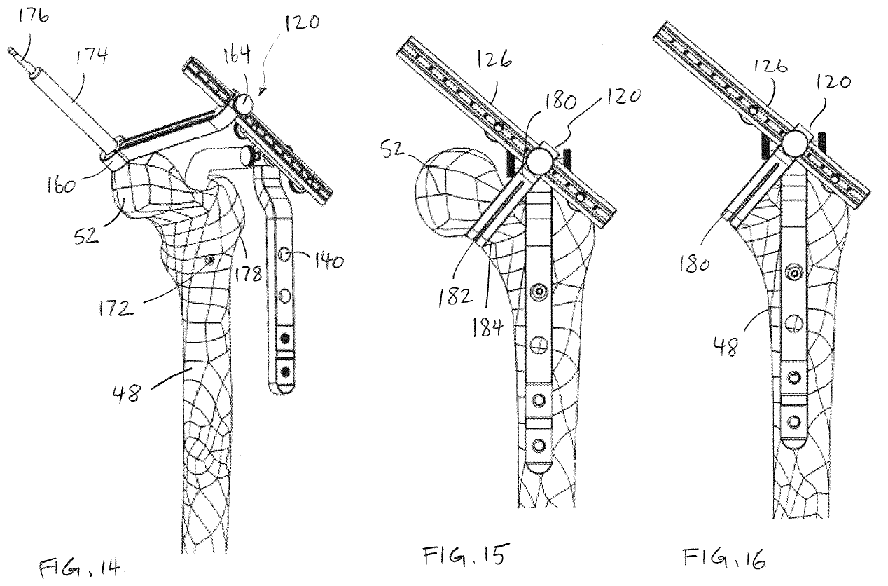

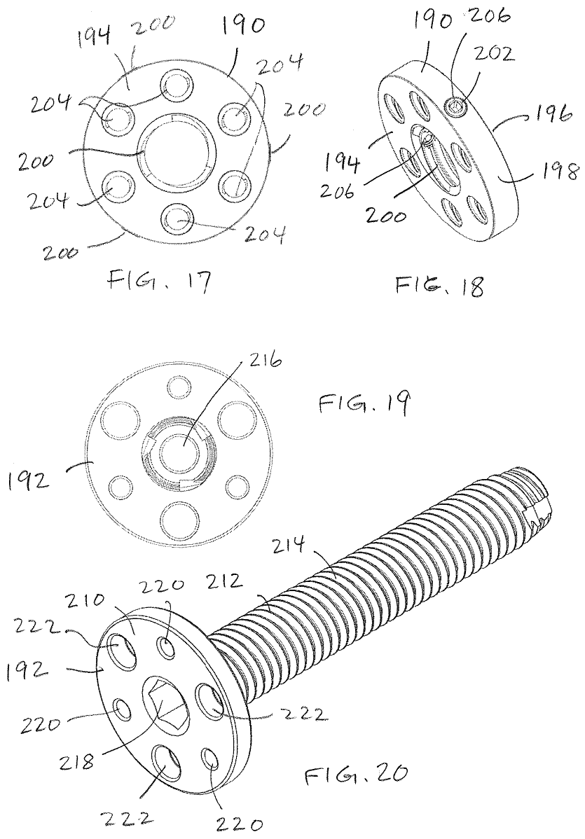

Turning to FIGS. 17 through 20, a bone anchor 190 and reamer guide 192 are then provided. In one embodiment, the bone anchor 190 is disc-shaped, includes a front face 194, a rear face 196, an outer periphery 198 extending between the front and rear faces 194, 196 and which defines a circumference, an axial threaded opening 200 passing through the front and rear faces, three set screw holes 202 equidistantly spaced-apart (at 120°) about the outer periphery 198 and extending radially from the outer periphery 198 into the axial threaded opening 200, and six locking screw holes 204 equidistantly spaced-apart (at 60°) about a center axis of the axial threaded opening) and extending parallel to the threaded opening 200. Set screws 206 are provided in the set screw holes 202.

In one embodiment in association with bone anchor 190, the reamer guide 192 includes a head 210 and a shaft 212. The head 210 is a flat disc-shaped structure having the same circumference as the bone anchor 190. The shaft 212 has threads 214 and defines a cannulated bore 216. The head 210 includes a hex-shaped driver recess 218 (or other driver engagement, including, for example, a threaded recess) that communicates with the bore 216. The head 210 also includes three smaller holes 220 spaced 120° apart, and three relatively larger holes 222 spaced at 120° apart, such that the center of one of the smaller or larger holes is 60° apart from another.

Turning to FIG. 21, the shaft 212 of the reamer guide 192 is threadedly engaged into the threaded opening 200 of the bone anchor 190, and advanced until the head 210 seats against the front face 194 of the bone anchor 190, and the holes 220, 222 in the head 210 of the reamer guide 192 align with the face screw holes 204 in the bone anchor 190.

Then, as shown in FIG. 22, the shaft 212 of the reamer guide 192 is inserted through the drilled hole in the femoral neck until the shaft 212 passes through the angled hole 96 (FIGS. 1, 2 and 3) in the nail 20 and the rear face 196 of the bone anchor 190 is flush with the cut surface 230 of the neck. A driver can be coupled to the driver recess 218 to advance the reamer guide as necessary. Referring to FIG. 23, the larger holes 222 of reamer guide 192 are used as guides for a drill bit 232 through at least three of the six face screw holes 204 of the bone anchor 190 (FIG. 17). (The reamer guide 192 can be rotated relative to the bone anchor 190 to drill more than three holes.) Then locking bone screws (not shown) are advanced to secure the bone anchor 190 at the remaining femoral neck bone 184.

Turning now to FIG. 24, a distal screw attachment guide 234 is attached to the anterior guide 124 of the jig 120. The distal screw attachment guide 234 is a curved bar with a first hole 236 and a second hole 238 at opposite ends of the bar. The holes 236, 238 have central axes which are oriented 90° to each other. The end of the distal screw attachment guide 234 with hole 236 is rigidly assembled at the threaded hole 136 (FIG. 23) of the anterior guide 124 with a set screw 240 such that the second hole 238 is axially aligned with one of the first set of screw holes 98 in the nail 20 (FIGS. 3, 4, and 5). A drill guide 242 and drill 244 are utilized to drill holes through the near cortex, the one of the first screw holes 98 in the nail 20, and the far cortex. A cortical screw 246 (FIG. 25) is inserted through the bone 48 and the one of the first screw holes 98 in the nail 20. The process can be repeated for the other one of the set of first screw holes 100 of the nail. The distal screw attachment guide 234 is then removed from the jig 120. Referring to FIG. 25, a drill bit 248 is then inserted through the reamer guide 192 and a hole is drilled through the lateral cortex of the femur 48 in alignment with the bore of the reamer guide.

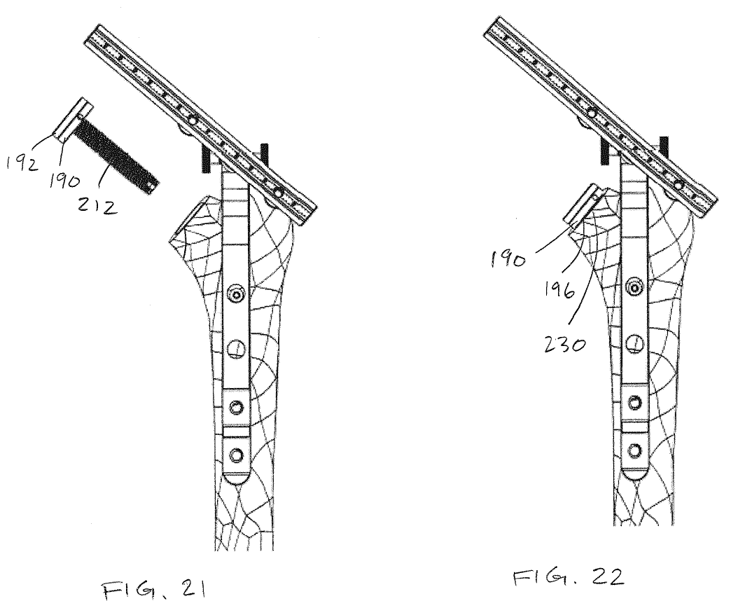

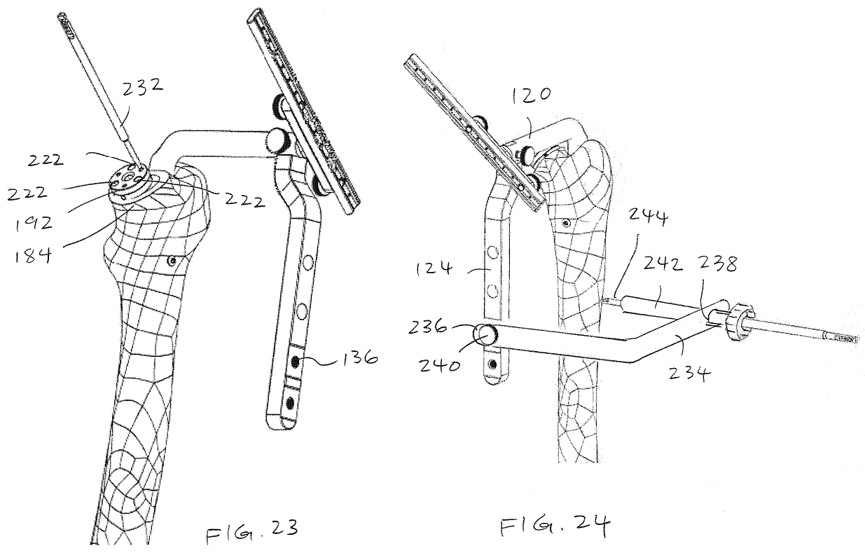

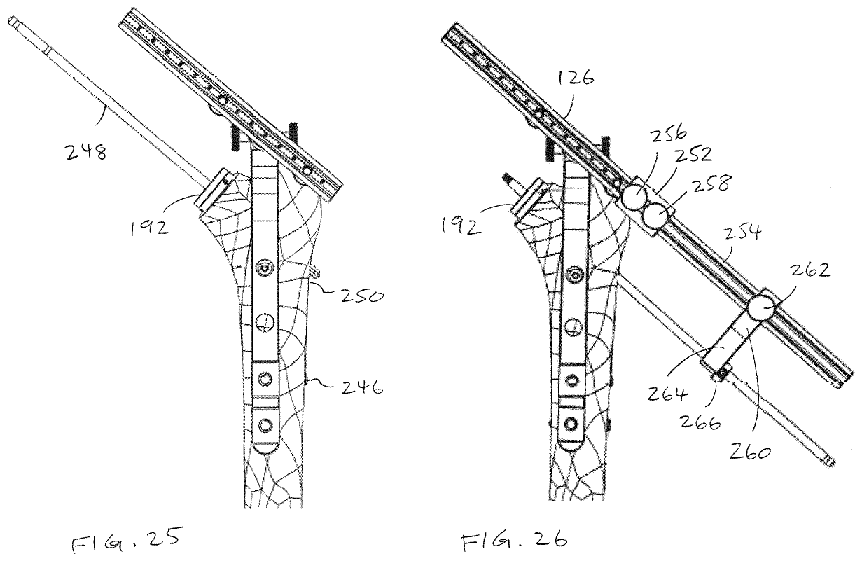

Turning to FIG. 26, a rail connector 252 and rail extension 254 are attached to the rail 126. The rail connector 252 is extends about an end of the rail 126, includes two threaded screw holes each with a locking screw 256, 258. One end of the connector is slid over the lateral side of the rail 126 and secured with set screw 256. The rail extension 254 is inserted into the opposite end of the connector and secured with the set screw 258 to hold the rail extension 254 axially aligned with the rail 126. A lateral guide 260 is advanced over the rail extension 254 and securable to the rail extension 254 with a set screw 262. The lateral guide 260 includes an arm 264 extending from the rail extension 254 at a predefined angle, and terminating in a guide ring 266. The guide ring 266 is sized to stably receive a reamer shaft 268 therethrough. The reamer shaft 268 is oriented by the guide ring 266 of the lateral guide 260 along an axis that extends through the shaft of the reamer guide 192. Once the reamer shaft 268 extends through the reamer guide 192, a reamer adapter 270 and reamer head 272 can be secured to the reamer shaft 268, as shown in FIGS. 27 and 28.

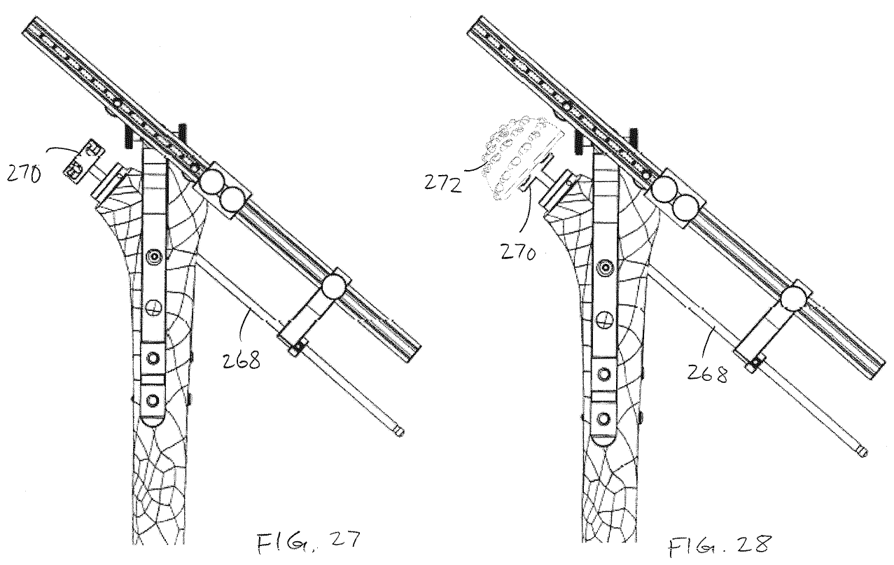

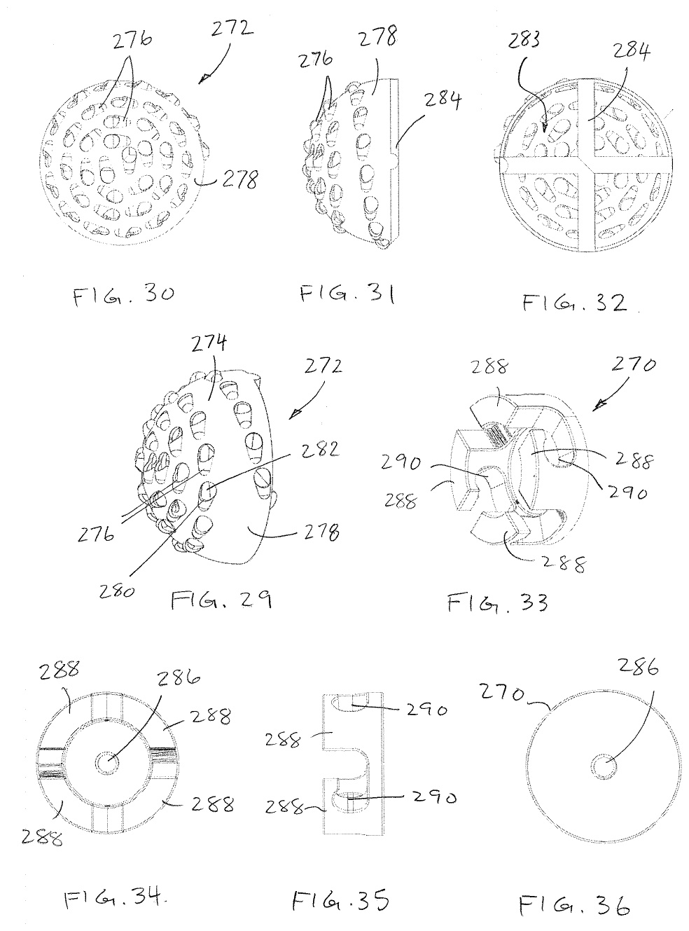

Referring to FIGS. 29 through 32, the reamer head 272 is a hollow hemispherical body 274 defining cutting heads 276 generally spirally arranged (FIG. 30) on its hemispherical outer surface 278. Each of the cutting heads 276 is defined by a sharp cutting edge 280 leading to an opening 282 that permits removal of reamed bone to the interior 283 of the reamer. A crossbar 284 is provided over the opening of the hollow body at its largest diameter. Turning to FIGS. 33 through 36, the reamer adapter 270 has a small opening 286 adapted to engage the reamer shaft 268, and a distal arrangement of four bayonet locks 288 at 90° separation and defining grooves 290 to capture portions of the crossbar 284. The bayonet locks 288 can be positioned relative to the crossbar 284 of the reamer head, and reamer head 272 and reamer adapter 270 rotated relative to each other such that the bayonet locks 288 engage the crossbar 284. Such engagement occurs when the reamer shaft 268 is rotated in a direction that results in the cutting heads 276 reaming the acetabular socket.

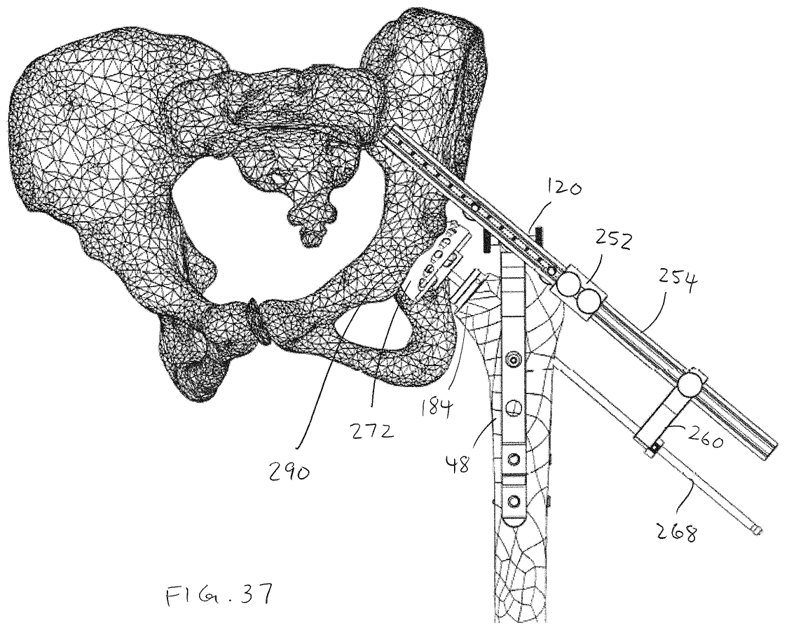

The femur is brought into alignment by internally rotating and adducting the hip joint. The reamer shaft 268 and reamer head 272 are then mechanically rotated to ream the acetabular socket 290, as shown in FIG. 37. The socket is preferably reamed in 1 mm increments via the reamer shaft 268 extending through the neck 184 of the femur 48 until all of the subchondral bone in the acetabular socket is removed. The reamer head 272 and reamer adapter 270 are then removed. The rail connector 252, rail extension 254 and lateral guide attachment 260 are also removed from the jig 120.

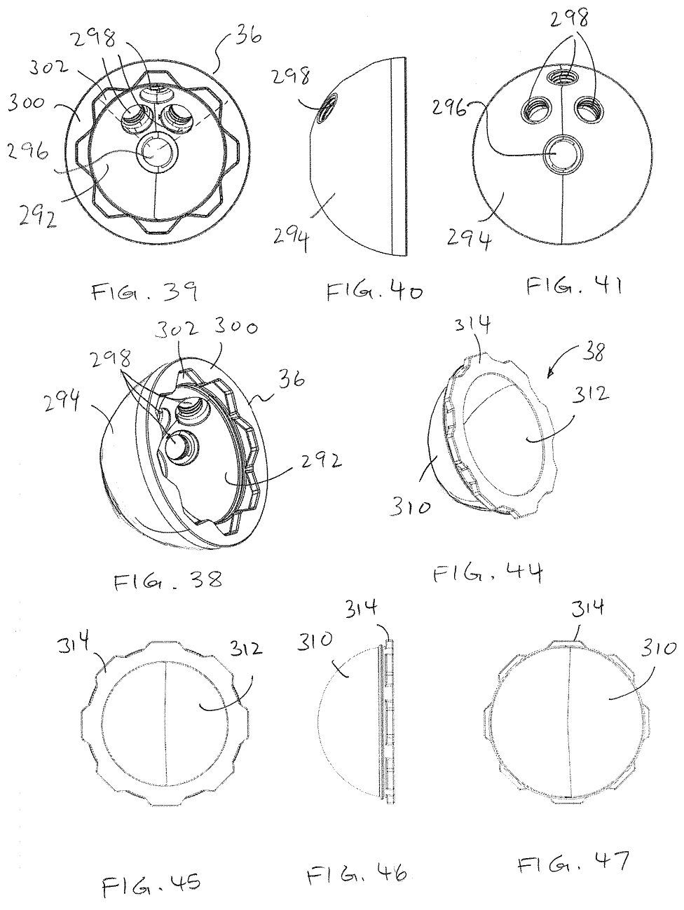

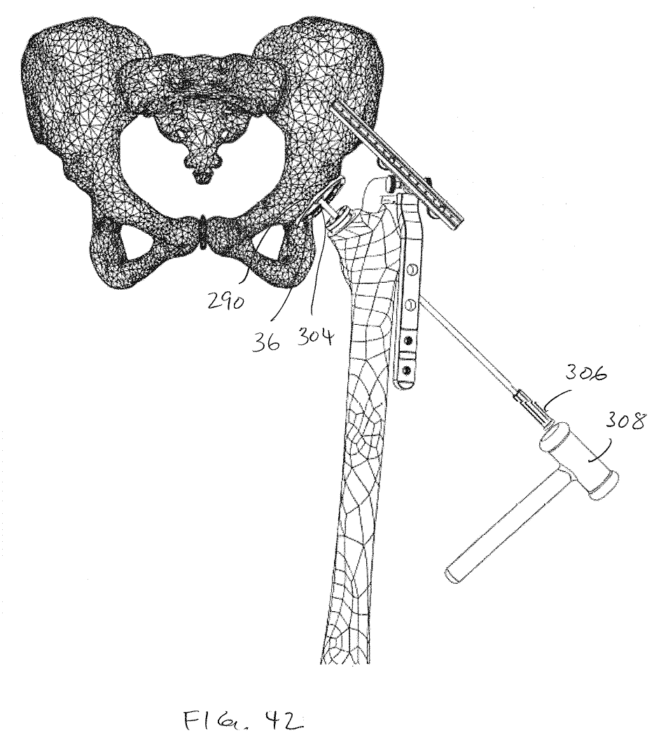

Turning to FIGS. 38 through 41, the acetabular cup 36 is selected based on prior measurements. The acetabular cup 36 is a generally hemispherical cup having a concave interior surface 292 and a convex exterior surface 294. The cup 36 includes a central threaded opening 296 and three fixed angle screw holes 298 displaced about the central opening 296. Preferably screw holes 298 are all provided within a single quadrant about opening 296 and oriented for suitable purchase in bone underlying the acetabulum. The cup 36 includes a rim 300 provided with a non-circular recess or recesses 302 extending circumferentially about the rim. The exterior surface 294 is preferably provided with a surface treatment to aid in bone fixation and integration.

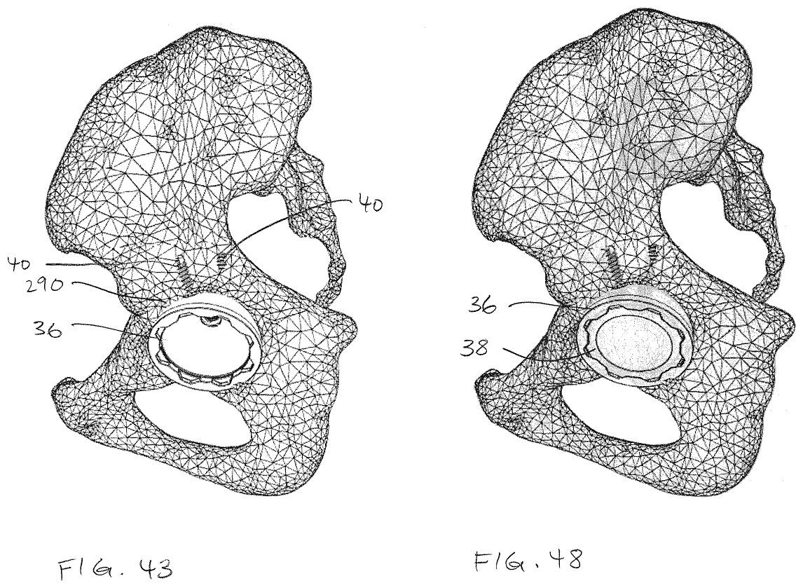

Turning to FIG. 42, the acetabular cup 36 is inserted into the reamed acetabular socket 290. While proper leg position is maintained, a rod impactor 304 is attached to the distal end of the reamer shaft 268 and the rod impactor 304 is threadedly connected to the central opening 296 of the acetabular cup 36. Then, the acetabular cup 36 is seated flush in the acetabular socket 290 by applying slight impact force against an opposite end 306 of the reamer shaft, e.g., with a hammer 308. Turning to FIG. 43, once the cup 36 is seated in the acetabular socket 290, bone screws 40 are inserted through one or more of the three fixed angle screw holes 298 of the cup 36, as necessary, to secure the acetabular cup 36 in the socket 290. Caps (not shown) are inserted into the screw holes to cover empty screw holes and screw heads.

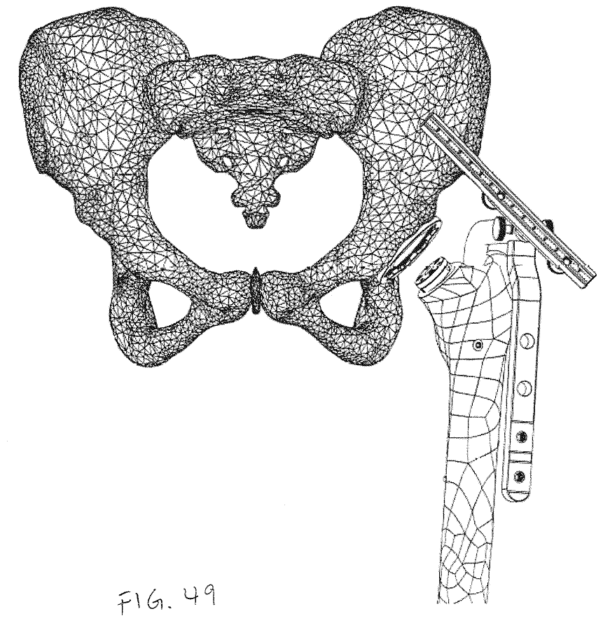

Turning to FIGS. 44 through 47, the cup liner 38 is then provided for the acetabular cup 36. The cup liner 38 has a hemispherical exterior surface 310 that conforms to the interior surface 292 of the acetabular cup 36, a hemispherical interior surface 312, and a lip 314 that seats against and rotationally interferes with the recess(es) 302 at the rim of the cup 36. The cup liner 38 is press fit and interference fit into the acetabular cup 36 (FIG. 48). The reamer guide 192 is unscrewed from the bone anchor 190 and removed the femur 48.

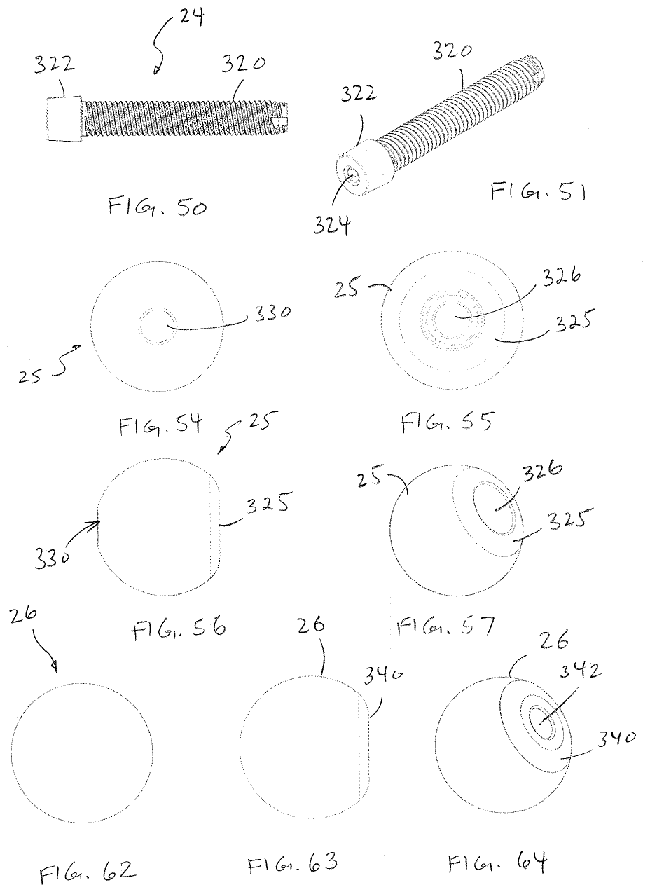

Referring to FIGS. 49 and 50, the neck screw 24 is provided and includes a threaded shaft 320 mating with the axial threaded opening 200 of the bone anchor 190, and a tapered seat 322 having an axial driver recess 324. The neck screw 24 with appropriate length of the shaft 320 is selected based on the radiographs or fluoroscopic images of the patient. Turning to FIG. 51, the shaft 320 of the neck screw 24 is threadedly advanced through the axial threaded opening 200 of the bone anchor 190, into the femur 48, and through the angled hole 96 of the nail 20 (FIGS. 3-5).

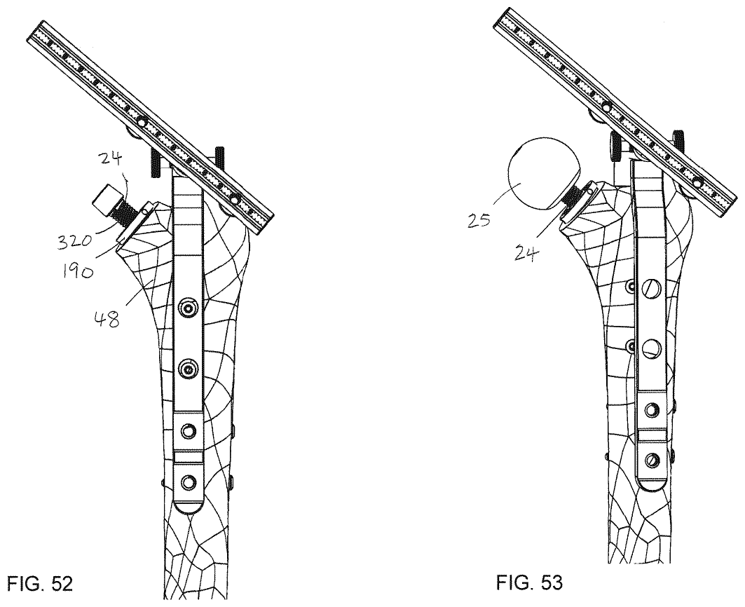

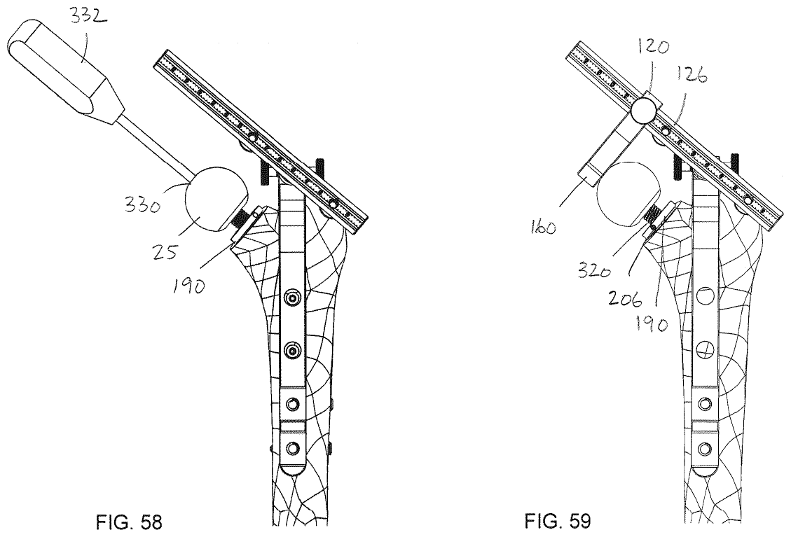

A trial femoral head 25 is then provided to the tapered seat 322 of the neck screw 24. (FIG. 52). Referring to FIGS. 53 through 56, the trial femoral head 25 is generally spherical, having a flat 326 on one side defining entry to a tapered recess 328 adapted to receive the seat 322, and an axial opening 330 permitting access to the driver recess 324 in the tapered seat 322 of the neck screw 24. Turning to FIG. 57, the longitudinal position of the trial femoral head 25 can be adjusted by rotating the neck screw 24 at the driver recess 324 through the axial opening 330 with a driver 332 to longitudinally displace the neck screw 24 relative to the bone anchor 190. The neck screw 24 is rotated to adjust the trial femoral head 25 to match the preoperative ICD measurement 60 (FIG. 2). Turning to FIG. 59, the apex ring 160 can be reattached to the rail 126 to aid in positioning; i.e., by using scale indicia thereon. The trial femoral head 25 is placed into the acetabular cup liner 38 to check for stability. If the surgeon notes any undesirable instability, preferably after ensuring the trial femoral head 25 is set at the correct ICD measurement 60, the joint can be checked and the neck screw 24 can be rotated to adjust the longitudinal position of the trial femoral head 25. Once the longitudinal position of the neck screw 24 and the size of the trial femoral head 25 is determined, the set screws 206 about the periphery of the bone anchor 190 are advanced and tightened against the threaded shaft 320 of the neck screw 24 to secure the longitudinal position of the neck screw 24 relative to the bone anchor 190. The guide jig 120 is removed from the proximal end of the nail 20. Turning to FIGS. 60, the nail set screw 34 is inserted through the proximal end of the nail 20 where the guide jig was coupled and driven into contact with the threaded shaft 320 of the neck screw 24. Then, referring to FIG. 61, the nail cap 35 is provided at the proximal end of the nail 20 to close the threaded bore 92 (FIGS. 3 and 6).

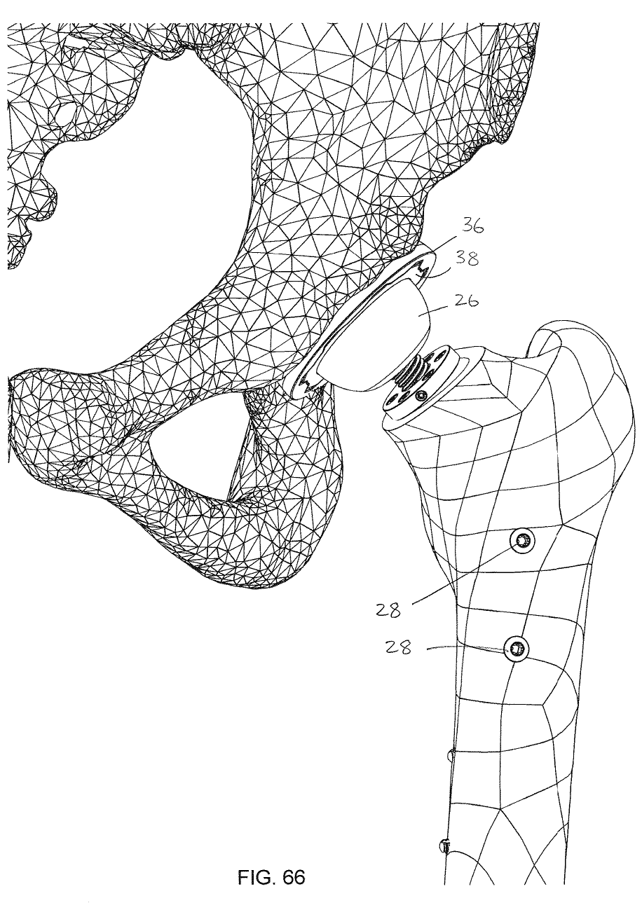

Referring to FIGS. 62 through 64, the prosthetic femoral head 26 corresponding to the selected trial femoral head 25 is provided. The prosthetic femoral head 26 has a spherical shape, except for a flattened portion 340 defining a tapered recess 342 sized to closely receive the tapered seat 322. As shown in FIG. 65, the prosthetic femoral head 26 is secured on the tapered seat 322 of the neck screw 24, preferably using a head impactor 350 and the mallet 308. Turning to FIG. 66, the prosthetic femoral head 26 is then inserted into the acetabular cup liner 38 in the acetabular cup 36 and proper placement, alignment, anteversion, and stability are confirmed. Anteversion is the angle of the femoral head forward. That is, the angle of the axis of the neck screw 24 and projected femoral head 26 with respect to a vertical frontal plane of the standing patient. The anteversion angle is normally about 15°±3°.

In a conventional hip prosthetic system, a stem portion of the prosthesis that is implanted in the proximal end of the femur is non-circular to prevent rotation of the stem. In addition, the angle of the head seat is determined at the time the surgeon broaches the femoral canal, which is done after the main native reference measurements are made and the femoral head is removed. Once the non-circular stem shaft is introduced in the canal, it is impacted and commonly cemented in whatever angle it landed. If the anteversion angle is to be adjusted, this can only be done via prosthetic heads with offset sockets. This requires higher an additional inventory of components, trial and error, and limited choices.

In distinction, the above-described system does not require offset sockets or is it limited by the initial angle by which the nail is implanted into the medullary canal. The rotational position of the nail is determined according to the native head before it is cut off. The guidance for the rotational orientation of the nail 20 is provided by the apex ring 60 which is seated at the top of the apex of the natural femoral head 52. Then, the nail 20 is secured via a cortical screw 28 inserted into screw holes 98 and 102 in the nail 20. If it is later determined that the surgeon wishes to change the rotational orientation of the nail 20, even after the femoral head is cut off, to vary the anteversion, the implanted cortical screw 28 in screw holes 98 and 102 can be removed, the nail 20 can be rotated, and cortical screws 28 can be installed in the other remaining screw holes 100 and 104 in the nail.

Alternatively, if no adjustment to the angular rotation is required, all additional screws may be provided in the screw holes to further stabilize the nail 20 in the bone. Such decision to implant additional screws can be made before or after the implant jig is removed. If the decision is made after the jig is removed, the jig 120 may need to be temporarily re-installed relative to the nail 20.

The incision is closed.

While the above-described system is complete, as described, it is appreciated that various alternative components can be used to the same or advantageous effect, and that variations on the method of implantation can be can performed.

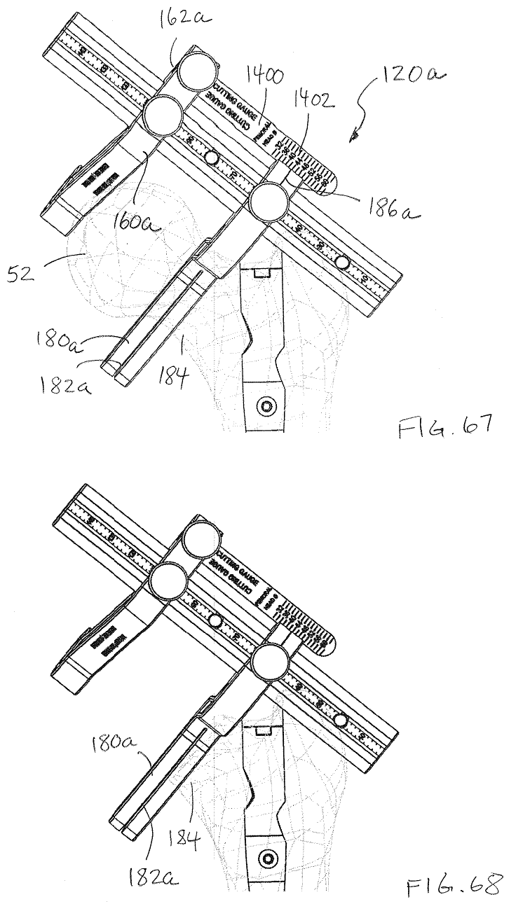

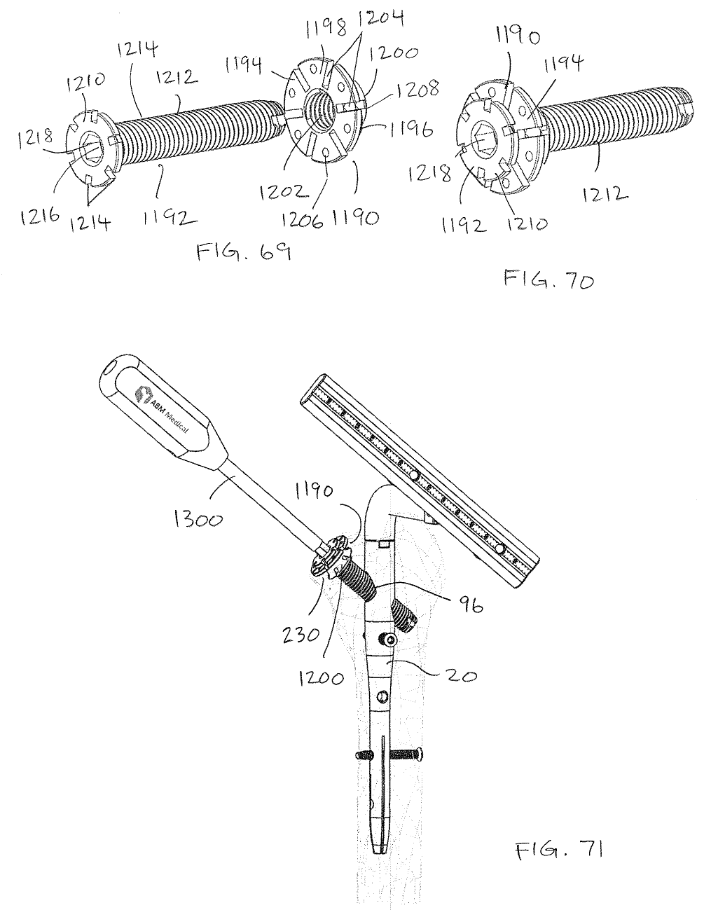

By way of example only, the jig 120a may carry one or more additional guides that assist in accurately measuring, orienting, and cutting bone at the site of implantation so that implants of appropriate size can be selected and to facilitate implantation of the components of the system into and relative to the bone. By way of example only, turning to FIG. 67, the apex ring 160a can include an attachment hole 162a. A cutting gauge 1400 can be secured to the apex ring at the attachment hole 162a. The cutting gauge 1400 includes linear-displaced indicia 1402 marked thereon. These markings 1402 indicate the location at which the cutting guide 180a should be referenced to cut the femoral neck 184 based on the diameter of the natural femoral head 52. For example, the cutting gauge 1400 may have indicia that represent between 32 and 60 mm. The indicia may cover a different range of sizes. The indicia may be in different or additional units. Using direct measurement or measurement from a radiographic image, the diameter of the natural femoral head 52 is obtained. Then, a reference line 186a on the cutting guide 180a is aligned with the associated indicia 1402 representing the measured diameter on the cutting gauge 1400. Referring to FIG. 68, this positions the cutting slot 182a of the cutting guide 180a at the intended location for a saw blade to cut through the femoral neck 184.

As another alternative for the system, a different bone anchor and reamer guide than that described above can be used with the system. Turning to FIGS. 69 and 70, bone anchor insert (bone anchor) 1190 and reamer guide 1192 are provided for use in conjunction with the system as generally described above. The bone anchor insert 1190 has a flat head 1198 and a short shaft 1200. The head 1198 has a front face 1194, a rear face 1196, an axial threaded opening 1202 passing through the front and rear faces 1194, 1196, and six radial slots 1204 extending through the head 1198 from the front face 1194 through to the rear face 1196 and dividing the head into respective sectors, with each sector including a hole 1206. The shaft 1200 has an external bone thread 1208.

The reamer guide 1192 includes a head 1210 and a shaft 1212. The head 1210 is flat and includes radial slots 1214. The shaft 1212 has bone engaging threads 1214 and defines a cannulated bore 1216. The head 1210 includes a hex-shaped driver recess 1218 (or other driver engagement including, for example, a threaded recess) that communicates with the bore 1216.

As shown in FIG. 70, the shaft 1212 of the reamer guide 1192 is threadedly engaged into the threaded opening 1200 of the bone anchor insert 1190, and advanced until the head 1210 seats against the front face 1194 of the bone anchor insert 1190.

Then, with reference to FIGS. 70 and 71, a driver 1300 is coupled to the driver recess 1218 of the reamer guide 1192 and driven to advance the shaft 1212 of the reamer guide 1192 through the drilled hole in the femoral neck until the shaft 1212 of the reamer guide 1192 passes through the angled hole 96 in the nail 20, the shaft 1200 of the bone anchor insert 1190 engages in the drilled hole in the femoral neck, and the rear face 1196 of the bone anchor insert 1190 is flush with the cut surface 230 of the neck.

Then, the drill bit 248 is used through the reamer guide 1192 to drill through the lateral cortex 250 (FIG. 72). After, as described with respect to the earlier embodiment, the drill bit is replaced by the reamer shaft, and a reamer head is attached, the acetabular surface is reamed and prepared. The cannulated bore 1216 of the reamer guide 1192 supports the reamer shaft. The previously described acetabular components are implanted. Then the reamer shaft is removed, and the reamer guide is removed from the bone anchor.

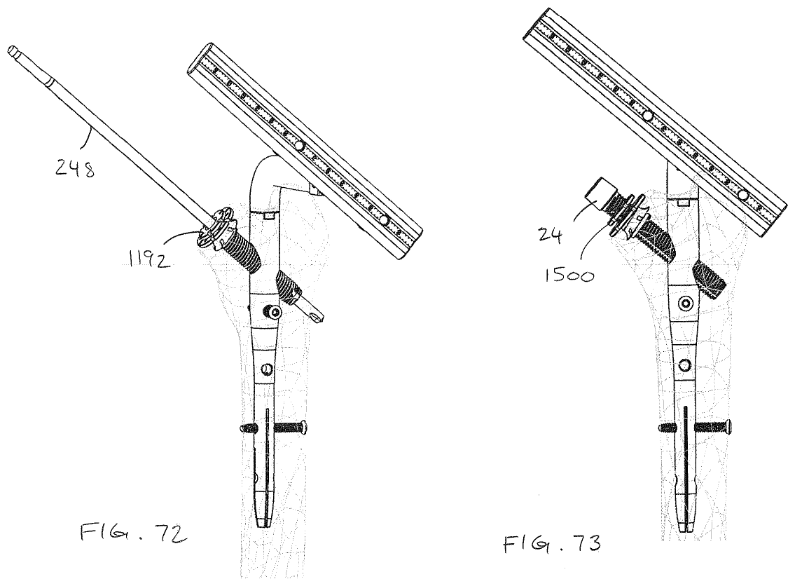

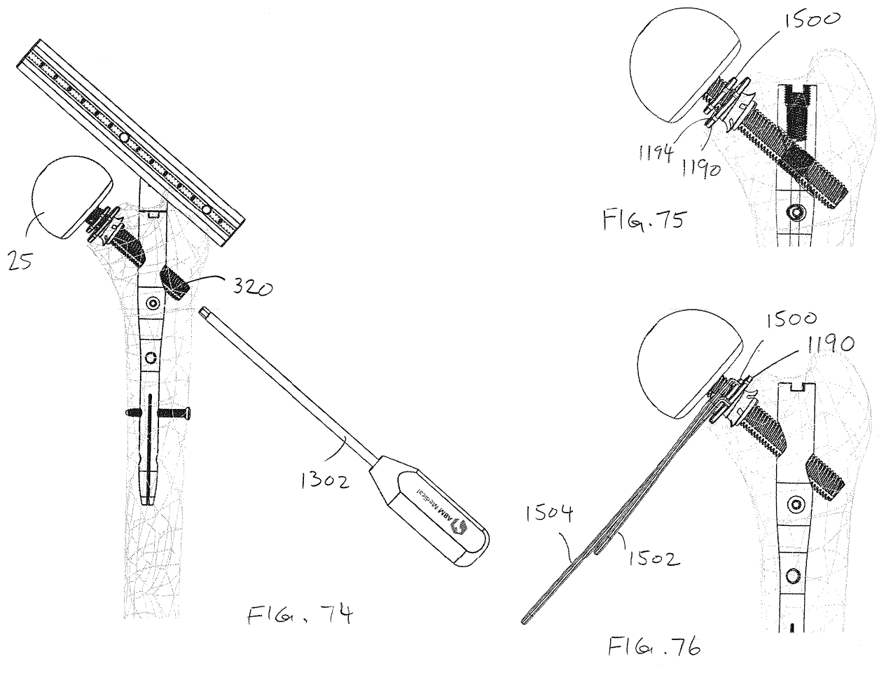

Turning to FIG. 73, a neck screw 24 of appropriate length is selected, and a jam nut 1500 is thread thereon. The jam nut 1500 is flat with radial slots and includes a central opening that threads onto the shaft of the neck screw. The jam nut 1500 is adapted to be thread down against the bone anchor to lock rotation of the neck screw relative to the bone anchor and thus fix the longitudinal position of the neck screw.

Referring to FIG. 74, as described in more detail above, a trial femoral head 25 is then coupled to the seat 322 of the neck screw 24 to check the diameter and longitudinal position of the trial head 25 on the neck screw 24. The longitudinal position can be adjusted from either the medial or lateral ends of the neck screw 24. From the medial end, adjustment is made at the seat 322 of the neck screw 24 via an opening in the trial 25 using driver 1300. From the lateral end, adjustment is made from an instrument 1302 inserted through the lateral cortex and into the shaft 320 of the neck screw 24.

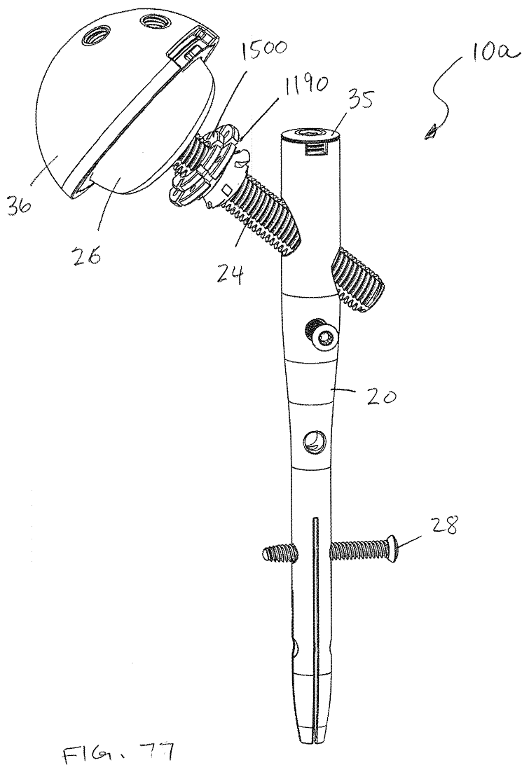

Once the neck screw 24 is at the correct longitudinal position, the trial 25 is removed, and the jam nut 1500 is threadedly tightened against the front face 1194 of the bone anchor insert 1190; i.e., moving the jam nut 1500 from the position shown in FIG. 75 to the position in FIG. 76. To facilitate the tightening, instruments 1502, 1504 are provided that engage with the respective radial slots of the jam nut 1500 and the bone anchor insert 1190 such that the jam nut 1500 can be tightly rotated into contact against the front face 1194 of the bone anchor insert 1190. Once the jam nut 1500 is tightened to secure the longitudinal position of the neck screw 24, an impactor is used to secure the femoral head implant 26 on the seat 322 of the neck screw 24. Other aspects of the implant system 10a, shown completed in FIG. 77, can be as previously described.

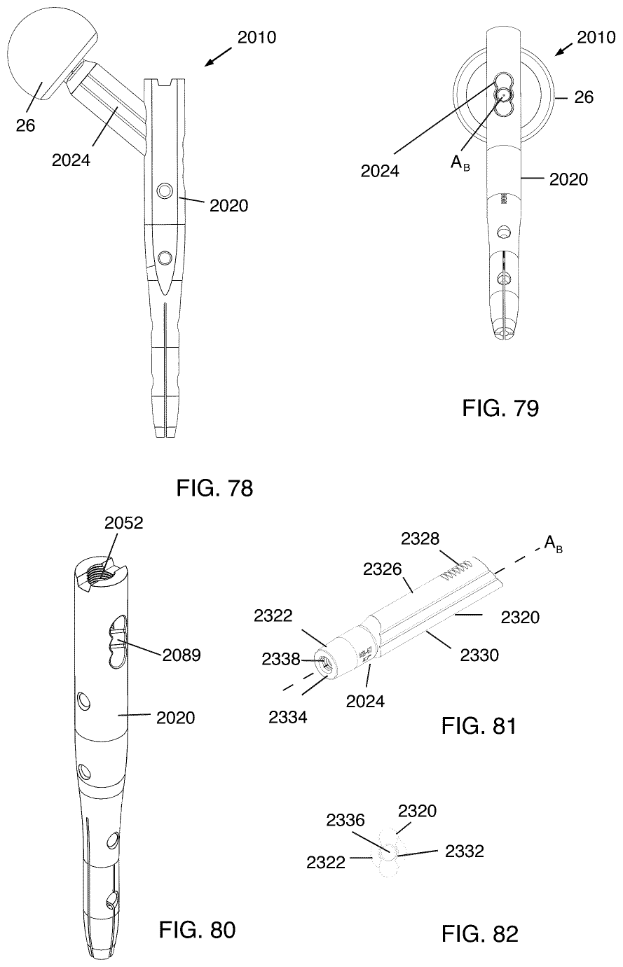

Turning to FIGS. 78 though 89, in another alternative system 2010, a neck component is a neck beam 2024 instead of a neck screw. The neck beam 2024 includes a body 2320 having a first end 2332, and a tapered seat 2322 having a second end 2334. The first and second ends 2332, 2334 lie along a beam axis A_{B}. The tapered seat 2322 is adapted to receive a femoral head trial component 25 and implant component 26. The body 2320 of the beam 2024 has a non-circular shape along its length such that it cannot rotate within an opening 2089 in the nail 2020, described below. In a preferred embodiment, the body 2320 has a tri-lobe or triple barrel shape causing it to be narrower and taller than the shaft 320 of the previously described neck screw 24 (FIGS. 50 and 51). In an embodiment, the barrel shape of the body 2320 has a loading height (across the three barrels) of 18 mm, whereas the preferred diameter of the neck screw in the prior embodiments is 15.5 mm. The relative dimensions provide greater loading strength for its cross-sectional area. The upper surface 2326 of the body 2320 of the neck beam 2024 is provided with a first locking structure such as a set of teeth 2328, and the lower surface 2330 is substantially smooth. The set of teeth 2328 includes at least one tooth, and more preferably a series of multiple teeth for accurate adjustability, as discussed below. The set of teeth 2328 may extend along only a portion of the upper surface 2326 or along the entire upper surface of the body 2320. Each of the first and second ends 2332, 2334 of the beam 2024 is provided with a coupling structure centered along the beam axis A_{B} for attaching an instrument. In one embodiment, the coupling structure at each end 2332, 2334 is a threaded hole 2336, 2338. In another embodiment, the first and second ends, each includes a bayonet connector with a J-groove. In yet another embodiment, the first and second ends, each includes an AO-type connector. Other connections are possible.

The nail 2020 of system 2010 includes a corresponding triple barrel slot 2089 adapted to closely receive and orient the neck beam 2024 at an intended neck axis angle, for example 120°, 125° or 130° (i.e., between 110°-140°) relative to the axis through the distal end of the nail 2020.

As described with the method above, a hole is drilled axially through the femoral neck. For the neck beam 2024, a guide (not shown) is used to drill a barrel-shaped opening. A barrel shaped reamer guide (not shown) is inserted through the femoral neck and into the nail and stabilized within the barrel slot 2089. The reamer guide has a driver attachment socket, for example, in the form of a threaded opening, that can be engaged by a tip of a threaded driver instrument and used to manipulate the reamer guide. Instruments are used to ream the acetabular cup through the reamer guide and then insert the acetabular components, as previously disclosed.

The neck beam 2024 is then inserted through the drilled hole in the femoral neck and into the barrel slot 2089 of the nail 2020 and advanced to the appropriate distance based on prior measurement of the anatomical distance for the hip joint.

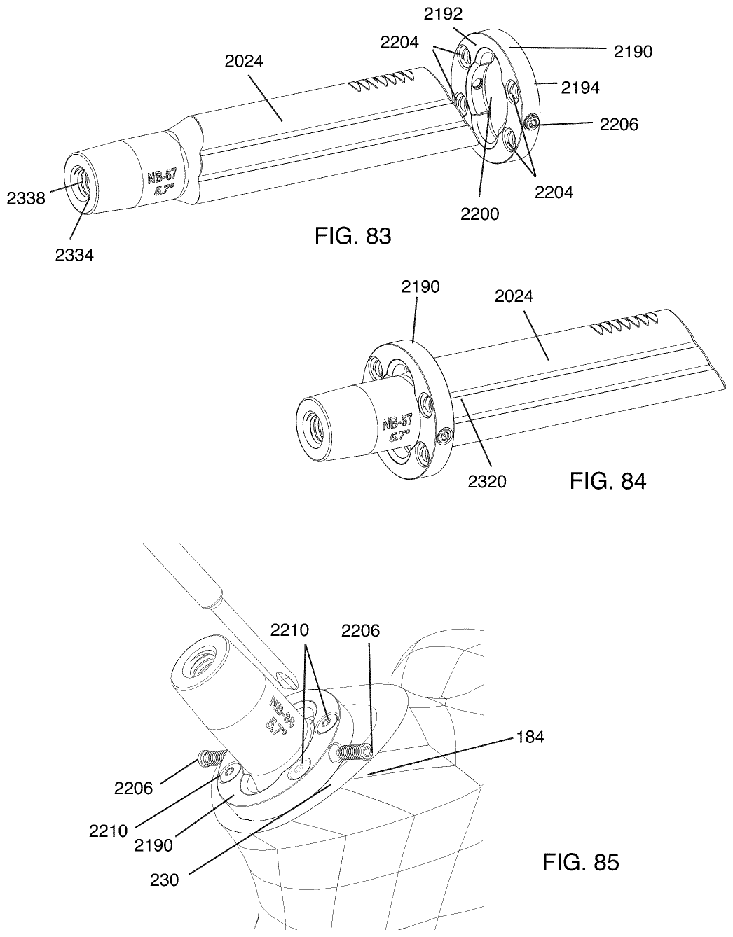

Turning to FIG. 83, a bone anchor in the form of a washer 2190 is provided. The bone anchor washer 2190 includes a tri-lobe central opening 2200, front and rear flat faces 2192, 2194, four screw holes 2204 extending through the front and rear faces, and a two radially arranged threaded locking screws 2206 extending from the outer periphery into the central opening 2200. Continuing at FIGS. 84 and 85, the central opening 2200 of the bone anchor washer 2190 is seated over the head end of the body 2320 of the neck beam 2024 and rear face 2914 is secured flush with the cut surface 230 of the neck femoral neck using bone screws 2210 installed through the four screw holes 2204 (FIG. 85).

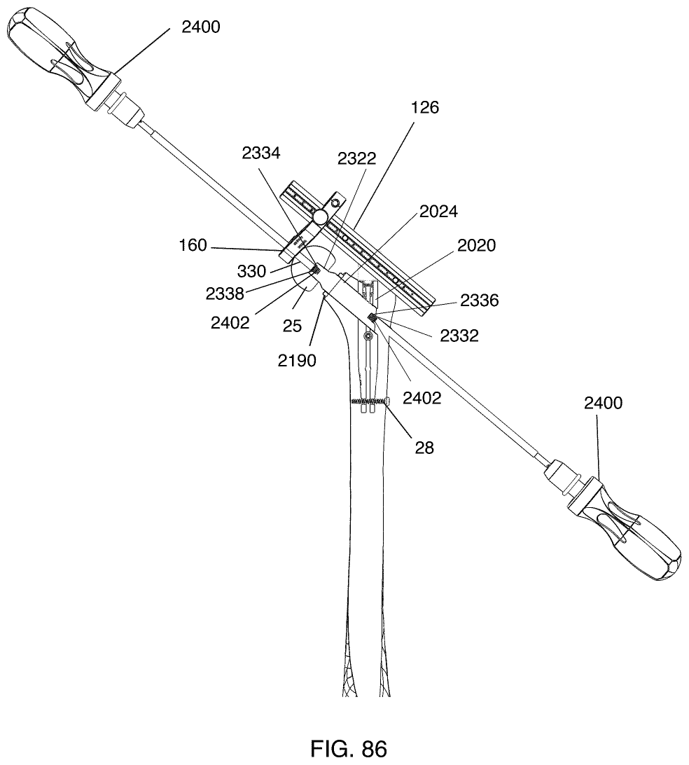

Turning to FIG. 86, the femoral head trial 25 is pushed onto the tapered seat 2322 of the neck beam 2024. The rail 126 is attached to the nail 2020 and the apex ring 160 is attached to the rail 126. A driver instrument 2400 is provided for attachment with the one of the first and second ends of the neck beam 2024. The driver instrument 2400 has an end 2402 adapted to removably engage with each of the first and second ends of the neck beam. In the illustrated embodiment, the working end of the driver instrument has a threaded end 2402 that is sized to threadedly couple with each threaded holes 2336 and 2338. Depending on which approach is better accessible to the beam 2024, the working end of the driver instrument 2400 is either advanced from a medial side through the hole 330 in the femoral head trial 25 and engaged in the threaded hole 2336 at the first end 2332 of the neck beam 2024, or, from a lateral approach, attached at the threaded hole 2338 in the second end 2334 of the neck beam 2024. The neck beam 2024 is then displaced relative to the nail 2020 to set the position of the femoral head trial 25 relative to the apex ring 160 according to prior measurements. The neck beam is temporarily locked in position relative to the beam by advancing the locking screws 2206 from the bone anchor 2190 into contact with the neck beam 2024 (FIG. 85).

Then, the femoral head trial 25 is test fit within the implanted acetabular cup liner 38. If any longitudinal adjustments are required to the position of the femoral head trial 25, the locking screws 2206 can be loosened, the driver 2400 can be reattached to the neck beam 2024, and the driver 2400 can be manipulated to longitudinally displace the neck beam 2024 relative to the nail 2020. Once a satisfactory position of the neck beam 2024 is determined, the locking screws 2206 are re-tightened to hold the position of the neck beam 2024 relative to the bone anchor 2190 and the nail 2020. Similarly, if while checking the fit it is determined that an anteversion adjustment to the implant is required, such adjust can be made by removing the bone anchor 2190 and neck beam 2024, loosening the bone screws 28 in the nail 2020, rotating the nail 2020 by a required degree angle for correct anatomical fit, resecuring the nail 2020 relative to the femur, redrilling the hole in the femoral neck for the re-oriented neck beam 2024, as necessary, reinstalling the bone anchor, and proceeding with another test fit of the femoral head trial 25, generally all in accord with description above.

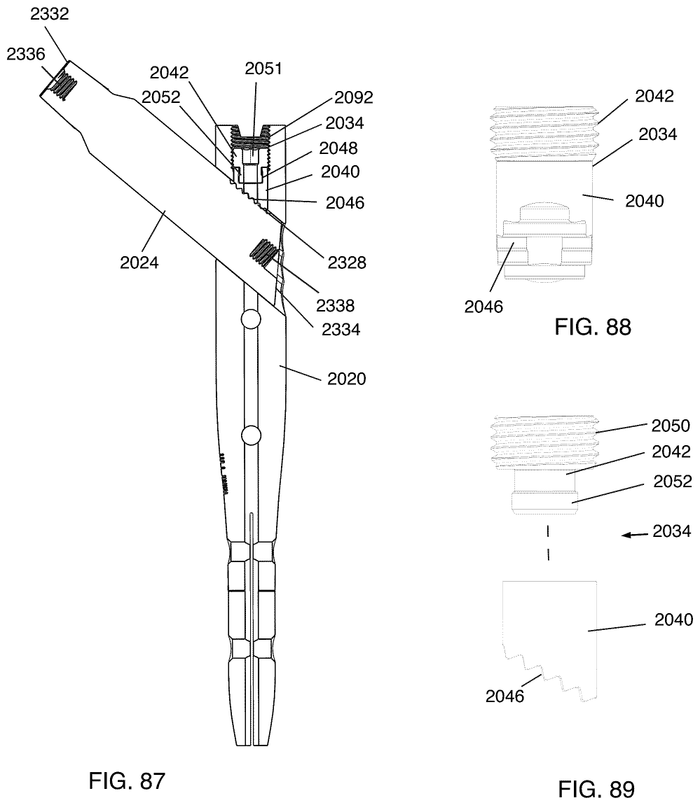

Referring to FIGS. 87 through 88, once the nail 2020 and neck beam 2024 are confirmed being in the correct position, the apex ring 160 and rail 126 are removed from the proximal end of the nail 2020. A set screw assembly 2034 is provided for the threaded bore 2092 at the proximal end of the nail 2020 to lock the neck beam 2024 in position relative to the nail 2020. The set screw assembly 2034 includes a lower tip element 2040 and an upper threaded head element 2042 coaxially positioned and rotatable relative to each other. The tip element 2044 is cylindrical and has a second locking structure such as a distal set of teeth 2046 arranged along an angle, and an upper receiver 2048. The set of teeth 2046 includes at least one tooth, and preferably multiple teeth for secure engagement with teeth 2328. The angle of the teeth 2046 is preferably within the neck axis angle. Different tip elements 2040 may be provided with teeth 2046 at each of the neck and triple barrel slot angles; alternatively, a single tip element 2040 may have teeth 2046 at an angle, size, and/or configuration that will operate for all of the neck and triple barrel slot angles. The threaded head element 2042 has an upper threaded portion 2050 with a driver receiver 2051, and a lower base 2052 that is received in the upper receiver 2048 of the tip element. The head element 2042 may engage the upper portion 2050 of the nail 2020 in a different manner to provide linear force on the tip element 2044 against the neck beam 2024, and maintain its locking position in relation to the nail 2020. For example, bayonet locks or other locking systems can be used. The tip element 2040 and head element 2042 are permitted to rotate relative to each other. The tip element 2040 is situated so that its distal set of teeth 2046 seat against the teeth 2046 of beam to resist movement of the beam relative to the nail 2020.

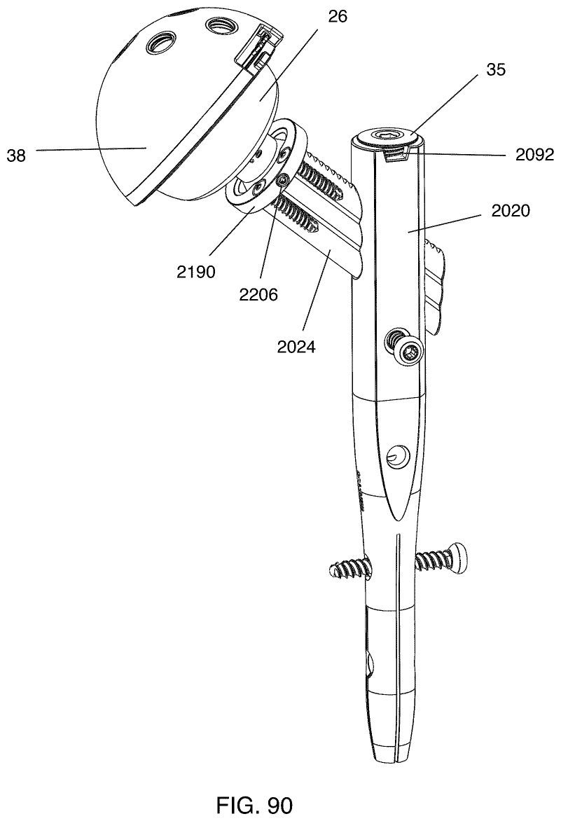

Turning to FIG. 90, the femoral head trial 25 (FIG. 86) is removed from the seat 2322 of the neck beam 2024 and replaced with the femoral head implant 26. Even after and while the joint is reduced, with the head implant 26 inserted into the acetabular cup liner 38, the fit of the joint can be adjusted. By loosening the locking screws 2206 at the bone anchor 2190 and the set screw assembly 2034 at the proximal end of the nail 2020 (FIG. 87), the neck beam 2024 can be displaced relative to the nail 2020. The driver can then be attached to the neck beam 2024 from a lateral approach and manipulated to longitudinally displace the neck beam 2024 relative to the nail 2020 while the head implant resides within the acetabular cup liner. This allows ideal determination and setting of fit. Once a satisfactory position of the neck beam 2024 is determined, the locking screws 2206 and set screw assembly 2034 are re-tightened to lock the neck beam 2024 relative to the bone anchor 2190 and the nail 2020, respectively. In addition, by adjusting in the reduced position, the use of the trial femoral head can even be eliminated. Further, a like process can be used to adjust the fit of the prosthetic femoral head 26 within anatomical structure such as an acetabulum while in a reduced position in a hemiarthroplasty procedure.

After final adjustment, an end cap 35 is inserted at the threaded bore 2092 at the proximal end of the nail 2020. Other aspects of the method, system and tools used for the implantation thereof are generally similar to other embodiments described above.

There have been described and illustrated herein embodiments of a total hip arthroplasty system and a method of total hip arthroplasty. While particular embodiments of the invention have been described, it is not intended that the invention be limited thereto, as it is intended that the invention be as broad in scope as the art will allow and that the specification be read likewise. As such, while several embodiments with different features have been described, it is specifically intended that features, components, fasteners, guides, instruments, and methods of one embodiment can be used with other embodiments, where appropriate. Thus, while particular guide components have been disclosed, it will be appreciated that methods for implanting the system described herein may be able to be carried out using alternative procedure, including a different order of steps. In addition, the implantable system has been described with respect to particular non-implanted guides, tools, and components therefor, it will be understood that the system and method of use are not limited to such guides and tools, and others can be used. Similarly, while the guides and tools have been described with respect to specific implantable prostheses, it is appreciated that the guides and tool are not limited thereto and could be used in association with other prosthetic systems. Further, while a total hip arthroplasty system is described, it is appreciated that the system can be used in part, such as for example without replacement of the acetabular bearing surface. In addition, where materials are disclosed, it is appreciated that other suitable materials having the requisite strength and biocompatibility can be used. It will therefore be appreciated by those skilled in the art that yet other modifications could be made to the provided invention without deviating from its spirit and scope as claimed.

1. A total hip arthroplasty system for a patient, comprising:
a) a medullary nail for insertion into a femoral canal of the femur, the nail having a proximal end, a distal end, a length between the proximal and distal ends, a longitudinal axis extending through the proximal and distal ends, a neck opening having a central axis extending at a neck angle between 110° to 140° relative to the longitudinal axis;
b) a neck beam having a body and a seat, and opposite first and second ends situated along a beam axis,
the body of the neck beam having a width and a height greater than the width,
the body of the neck beam having an upper surface of the body of the neck beam provided with an engagement structure,
the body of the neck beam supportable in the neck opening at the neck angle and longitudinally displaceable relative to the neck opening at the neck angle, and
each of the first and second ends having a tool engagement structure permitting the beam to be engaged by a tool and longitudinally displaced within the neck opening;
c) a locking assembly having
a first locking structure adapted to engage the proximal opening of the medullary nail, and
a second locking structure adapted to engage with the engagement structure on the body of the neck beam,
whereby the locking assembly is adapted to fix the longitudinal position of the neck beam within the neck opening of the medullary nail; and
d) a femoral head adapted to engage the seat of the neck beam.

2. The total hip arthroplasty system of claim 1, wherein:
the body of the neck beam defines a triple barrel shape, and
the neck opening defines a triple barrel shape that closely receives the body of the neck beam.

3. The total hip arthroplasty system of claim 1, wherein:
the engagement structure includes a plurality of teeth, and the first locking structure includes at least one tooth.

4. The total hip arthroplasty system of claim 1, wherein:
the proximal end of the medullary nail includes a threaded portion, and
the locking assembly includes a first component having an end oriented at a first angle and a second component with a threaded portion adapted to mate with the threaded portion, the first and second components are coaxial and rotatable relative to each other.

5. The total hip arthroplasty system of claim 1, further comprising:
a bone anchor having a central opening, a front face, a rear face, a plurality of screw holes extending through the front and rear faces, and at least one locking screw extending into the central opening,
the central opening sized and shaped to receive the body of the neck beam and
wherein when the body of the neck beam is positioned through the central opening of the bone anchor and the locking screw is tightened, the bone anchor washer is secured relative to the neck beam.

6. The total hip arthroplasty system of claim 5, wherein:
the body of the neck beam defines a triple barrel shape,
the neck opening defines a triple barrel shape, and
the central opening defines a triple barrel shape.

7. The total hip arthroplasty system of claim 1, wherein the distal end of the nail has at least one diametric split.

8. The total hip arthroplasty system of claim 1, wherein the nail is cannulated.

9. The total hip arthroplasty system of claim 1, wherein the nail is straight.

10. The total hip arthroplasty system of claim 1, wherein the neck beam is solid.

11. The total hip arthroplasty system of claim 1, wherein the tool engagement structure is an internal thread.

12. The total hip arthroplasty system of claim 1, further comprising:
an acetabular cup assembly for articulation with the femoral head.

13. A hip prosthesis system, comprising:
a) a medullary nail for insertion into a femoral canal of the femur, the nail having a proximal end, a distal end, a length between the proximal and distal ends, a longitudinal axis extending through the proximal and distal ends, a neck opening having a central axis extending at a neck angle relative to the longitudinal axis;
b) a neck component having a body and a seat,
the body of the neck component having a width and a height greater than the width,
the body of the neck component supportable in the neck opening at the neck angle and longitudinally displaceable relative to the neck opening at the neck angle;
c) a locking assembly adapted to secure the neck component in position relative to the medullary nail; and
d) a bone anchor having a central opening, a front face, a rear face, a plurality of screw holes extending through the front and rear faces, and at least one locking screw extending into the central opening, the central opening sized and shaped to receive the body of the neck beam,
wherein when the body of the neck component is positionable through the central opening of the bone anchor and the locking screw is tightened, the bone anchor is secured relative to the neck component.

14. The hip prosthesis system of claim 13, wherein:
the body of the neck component defines a triple barrel shape, and
the neck opening defines a corresponding triple barrel shape adapted to closely receive the body of the neck component.

15. The hip prosthesis system of claim 13, wherein the rear surface of the bone anchor is flat.

16. The hip prosthesis system of claim 13, wherein the bone anchor is disc-shaped.

17. The hip prosthesis system of claim 13, wherein the neck angle is between 110° to 140° relative to the longitudinal axis.

18. The hip prosthesis system of claim 13, wherein:
the body of the neck component includes an upper surface, and
the locking assembly includes a first locking structure adapted engage the medullary nail, and second locking structure adapted to engage the upper surface of the body of the neck component.

19. The hip prosthesis system of claim 13, wherein the nail is straight.

20. The hip prosthesis system of claim 13, further comprising:
a femoral head adapted to engage an end of the neck component; and
an acetabular cup assembly for articulation with the femoral head.

21. A method of setting a position of a prosthetic femoral head during a hip arthroplasty, comprising:
a) cutting the femoral head off the femoral neck to define a flat bone surface;
b) providing a bone anchor having a central opening extending between the front and rear faces;
c) securing the rear face of the bone anchor to the flat bone surface;
d) providing a neck component having a head seat and a body;
e) positioning the body of the neck component within the central opening of the bone anchor;
f) locking the position of the neck component relative to the bone anchor; and
g) implanting the prosthetic femoral head on the head seat.

22. The method of claim 21, further comprising:
prior to locking, adjusting the longitudinal position of the neck component relative to the bone anchor.

23. The method of claim 21, wherein the body of the neck component is non-circular in cross-sectional shape, and the central opening in the bone anchor is a corresponding size and shape.

24. The method of claim 21, wherein the bone anchor includes at least one locking screw displaceable relative to the central opening, and the locking includes moving the at least one locking screw into interference with the body of the neck component.

25. A method of setting a position of a prosthetic femoral head during a hip arthroplasty, comprising:
a) implanting a nail within a medullary canal of a femur, the nail having an opening;
b) inserting a neck component through the opening in the nail;
c) providing a femoral head trial onto an end of the neck component, the femoral head trial having an opening axial with the neck component and communicating with the neck component;
d) measuring or testing the fit of the femoral head trial;
e) inserting an instrument through the opening in the femoral head trial, engaging the neck component, and longitudinally displacing the neck component relative to the nail with the instrument;
f) locking the position of the neck component relative to the nail; and
g) replacing the femoral head trial with a prosthetic femoral head.

26. The method of claim 25, wherein the neck component has a body with non-circular cross-sectional shape, and the longitudinally displacement occurs without rotating the nail.

27. The method of claim 25, wherein the instrument engages the neck component with a threaded coupling.

28. A method of setting a position of a prosthetic femoral head during a hip arthroplasty, comprising:
a) implanting a nail within a medullary canal of a femur, the nail having an opening;
b) inserting a neck component through the opening in the nail;
c) inserting a prosthetic femoral head onto the neck component;
e) positioning the prosthetic femoral head into a corresponding hip joint component in a pelvis, the femoral head rotatable relative to the corresponding hip joint component;
f) engaging the neck component with an instrument, and longitudinally displacing the neck component relative to the nail with the instrument; and
g) locking the position of the neck component relative to the nail.

29. The method of claim 28, wherein the longitudinally displacing of the neck component is performed while the prosthetic femoral head is positioned within the corresponding hip joint component.

30. The method of claim 29, wherein the corresponding hip joint component is a prosthetic acetabular cup.

31. The method of claim 29, wherein the corresponding hip joint component is an acetabulum.

32. A method of implanting a total hip prosthesis, comprising
a) implanting a medullary nail into a femoral canal of a femur, the nail having a proximal end, a distal end, a longitudinal axis extending through the proximal and distal ends, and a neck hole having a central axis extending at an angle between 110° to 140° relative to the longitudinal axis;
b) cutting a femoral head off a femoral neck of the femur to form a cut-off femoral neck;
c) inserting a neck component having a head seat and a body, the body passing through the neck hole of the medullary nail;
d) implanting an acetabular cup assembly;
e) placing a femoral head on the neck seat of the neck component; and
f) inserting the femoral head into the acetabular cup assembly.

33. The method of claim 30, further comprising:
implanting a bone anchor having a central opening at the cut-off femoral neck, the neck component passing through the central opening of the bone anchor.

34. The method of claim 33, further comprising:
checking anteversion of the femoral head into the acetabular cup assembly;
if the anteversion needs to be adjusted, removing the femoral head, the neck component, and bone anchor;
then rotating the medullary nail in the femoral canal by an angle suitable to correct anteversion; and
reimplanting the bone anchor, the neck component, and the femoral head.

35. The method of claim 33, further comprising:
adjusting a longitudinal displacement of the neck component relative to the bone anchor by moving the body of the neck component relative to the central opening of the bone anchor; and
locking the longitudinal displacement of the neck component relative to the bone anchor.
